(19) United States
(12) Patent Application Publication
(10) Pub. No.: US 2025/0289896 A1
(43) Pub. Date: Sep. 18, 2025

(54) ANTI-CCR8 ANTIBODIES AND USES THEREOF

(71) Applicant: ANALYTICAL BIOSCIENCES SHANGHAI LIMITED, Shanghai (CN)

(72) Inventors: Xiaodong HUANG, Shanghai (CN); Mingjian FEI, Shanghai (CN); Qianting ZHAI, Shanghai (CN); Hao ZHANG, Shanghai (CN); Jun CAO, Shanghai (CN); Yanna MA, Shanghai (CN)

(21) Appl. No.: 18/860,940

(22) PCT Filed: Apr. 28, 2023
(86) PCT No.: PCT/CN2023/091639

§ 371 Date: Oct. 28, 2024

(30) Foreign Application Priority Data
Apr. 29, 2022 (WO) PCT/CN2022/090240

(51) Int. Cl. C07K 16/28; A61K 45/06

(52) U.S. Cl. CPC C07K 16/2866; A61K 45/06; C07K 2317/24; C07K 2317/41; C07K 2317/52; C07K 2317/565; C07K 2317/732; C07K 2317/76; C07K 2317/92

(57) ABSTRACT

Disclosed are an anti-CCR8 antibody or antigen-binding portion thereof; a pharmaceutical composition comprising the anti-CCR8 antibody or antigen-binding portion thereof and a pharmaceutically acceptable carrier; a method of treating a disease or condition in a subject in need thereof using the anti-CCR8 antibody or antigen-binding portion or the pharmaceutical composition; and use of the anti-CCR8 antibody or antigen-binding portion thereof in the manufacture of a pharmaceutical for treating a disease or condition.

### BACKGROUND

Immunotherapy is a rapidly advancing and very promising treatment for various forms of cancer, with many recent successes. However, some patients have limited or no response to current immunotherapies, and others exhibit relapse following initial responsiveness.

The human immune system includes checks and balances that serve to prevent an overactive immune system from harming the body. Regulatory T cells (Tregs) play an important role in maintaining a functional immune system by suppressing the immune responses. However, when Tregs infiltrate a tumor, they can block immune responses to the tumor. Therefore, there is a need to develop a more effective approach to selectively target tumor-infiltrating Tregs. Since CCR8 is preferentially expressed on tumor infiltrating Tregs, targeting CCR8 may be an effective therapeutic strategy to specifically modulate tumor-infiltrating Tregs in the tumor microenvironment (TME) and augment antitumor immunity.

### SUMMARY

The present disclosure provides anti-CCR8 antibodies which specifically bind to CCR8 and effectively inhibit tumor growth, and thus can be used for prevention or treatment of various cancers.

In a first aspect, the present disclosure provides an anti-CCR8 antibody or antigen-binding portion thereof comprising a heavy chain CDR1 having any one of the amino acid sequences set forth in SEQ ID NOs: 1-2, 56-60, 62, a heavy chain CDR2 having the amino acid sequence set forth in SEQ ID NO: 3, 63-67, 69-73, a heavy chain CDR3 having any one of the amino acid sequences set forth in SEQ ID NOs: 4-5, 74-78, 80-85, a light chain CDRI having any one of the amino acid sequences set forth in SEQ ID NOs: 6-11, 86-90, 92-93, a light chain CDR2 having the amino acid sequence set forth in SEQ ID NO: 12, 94-98, 100-102, and a light chain CDR3 having any one of the amino acid sequences set forth in SEQ ID NOs: 13-14, 103-107, 109-113.

In a second aspect, the present disclosure provides a pharmaceutical composition comprising the anti-CCR8 antibody or antigen-binding portion thereof according to the present disclosure and a pharmaceutically acceptable carrier.

In a third aspect, the present disclosure provides a method of treating a disease or condition in a subject in need thereof, comprising administering to the subject the anti-CCR8 antibody or antigen-binding portion according to the present disclosure or the pharmaceutical composition according to the present disclosure.

In a fourth aspect, the present disclosure provides use of the anti-CCR8 antibody or antigen-binding portion thereof according to the present disclosure in the manufacture of a pharmaceutical for treating a disease or condition.

### BRIEF DESCRIPTION OF THE DRAWING

FIG. 1 shows the binding titration of anti-CCR8 mAbs ChH2, H2A, H2B, H2C, H2G and H2I to TALL-1 cells with endogenous human CCR8 on the cell surface, wherein the X-axis denotes concentration of the anti-CCR8 mAb, and Y-axis denotes mean fluorescent intensity (MFI).

FIG. 2 shows the binding titration of anti-CCR8 mAbs ABC047-B1.IgG1.SDIE, ABC048-B1.hu11.IgG1.SDIE, ABC049-B1.hu12.IgG1.SDIE, ABC050-B1.hu13.IgG1.SDIE, ABC051-B1.hu14.IgG1.SDIE to 293T cells with overexpressed human CCR8.

FIG. 3 shows the binding titration of anti-CCR8 mAbs ABC025-H2G.SDIE, ABC047-B1.IgG1.SDIE, ABC137-H2.hu12.IgG1.SDIE, and ABC138-H2.hu13.IgG1.SDIE to 293T cells with overexpressed human CCR8, wherein the X-axis denotes concentration of the anti-CCR8 mAb, and Y-axis denotes MFI.

FIG. 4 shows the binding titration of anti-CCR8 mAbs ABC025-H2G.SDIE, ABC048-B1.hull.IgG1.SDIE, ABC138-H2.hu13.IgG1.SDIE, ABC078.AF, and ABC084.AF to activated Treg cells derived from human PBMC.

FIG. 5 shows the binding titration of anti-CCR8 mAbs ABC814, ABC815, ABC816, ABC817, ABC818, ABC819, ABC820, ABC821, ABC822, ABC823, ABC531, ABC138-H2.hu13.IgG1.SDIE, ABC080.chH2.SDIE, ABC026, and ABC078 to 293T cells overexpressing human or cynomolgus macaque CCR8, as well as Hut78 cell lines with endogenous CCR8, wherein the X-axis denotes logarithm values of anti-CCR8 mAb concentration, and Y-axis denotes GeoMean.

FIG. 6 shows the titration of anti-CCR8 mAbs ABC138-H2.hu13.IgG1.SDIE, ABC025-H2G.SDIE, and ABC080.chH2.SDIE to human PBMC-derived activated Treg cells, wherein the X-axis denotes concentrations of antibodies, and the Y-axis denotes MFI.

FIG. 7 shows the calcium flux assay on CHEMI-CCR8 cells with high human CCR8 expression on the cell surface, where the X-axis denotes time(s), and Y-axis denotes normalized chemotaxis ratios calculated by a formula: [F (fluorescence signal)−Fmin (minimal fluorescence signal representing baseline signal)]/Fmin. Different antibodies were used at a concentration of 66.7 nM.

FIG. 8 shows the calcium flux dose-titration assay on CHEM1-CCR8 cells with high human CCR8 expression on the cell surface, wherein the X-axis denotes concentration of the anti-CCR8 mAb, and Y-axis denotes normalized chemotaxis signal calculated by a formula: Max (maximal fluorescence signal achieved within 90 min upon CCL1 injection)−Min (minimal fluorescence signal representing baseline signal).

FIG. 9 shows ADCC killing of TALL-1 cells mediated by ChH2-AF or ChB1-AF.

FIG. 10 shows ADCC killing of Hut78 cells mediated by ABC024-H2G, ABC024-H2G.AF and ABC025-H2G.SDIE at a fixed concentration.

FIG. 11 shows ADCC killing of Hut78 cells mediated by ABC024-H2G.AF and ABC025-H2G.SDIE.

FIG. 12 shows ADCC killing of Jurkat-cynoCCR8 cells mediated by ABC089, ABC078, ABC138-H2.hu13.IgG1.SDIE, ABC822, and ABC1183.

FIG. 13 shows ADCC killing of Jurkat-hCCR8 mediated by ABC089, ABC078, ABC138-H2.hu13.IgG1.SDIE, ABC822, and ABC1183.

FIG. 14 shows ADCC killing of Hut78 cells mediated by ABC089, ABC078.AF, ABC138-H2.hu13.IgG1.SDIE, ABC822, and ABC1183.

### DETAILED DESCRIPTION OF CERTAIN EMBODIMENTS

As used in this application, except as otherwise expressly provided herein, each of the following terms shall have the meaning set forth below. Additional definitions are set forth throughout the application.

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth.

Unless defined otherwise, all technical and scientific terms used herein have the same meaning as commonly understood by one of ordinary skill in the art to which this disclosure is related.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the term “amount” or “level” is used in the broadest sense and refers to a quantity, concentration or abundance of a substance (e.g., a metabolite, a small molecule, a protein, an mRNA, a marker). When referring to a metabolite or small molecule (e.g. a drug), the terms “amount”, “level” and “concentration” are generally used interchangeably and generally refer to a detectable amount in a biological sample. “Elevated levels” or “increased levels” refers to an increase in the quantity, concentration or abundance of a substance within a sample relative to a control sample, such as from an individual or individuals who are not suffering from the disease or disorder (e.g., cancer) or an internal control. In some aspects, the elevated level of a substance (e.g., a drug) in a sample refers to an increase in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g., HPLC). “Reduced levels” refers to a decrease in the quantity, concentration or abundance of a substance (e.g., a drug) in an individual relative to a control, such as from an individual or individuals who are not suffering from the disease or disorder (e.g., cancer) or an internal control. In some aspects, a reduced level is little or no detectable quantity, concentration or abundance. In some aspects, the reduced level of a substance (e.g., a drug) in a sample refers to a decrease in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g, HPLC).

When referring to a protein, mRNA or a marker, such as those described herein, the terms “level of expression” or “expression level” in general are used interchangeably and generally refer to a detectable amount of a protein, mRNA, or marker in a biological sample. In some aspects, a detectable amount or detectable level of a protein, mRNA or a marker is associated with a likelihood of a response to an agent, such as those described herein. “Expression” generally refers to the process by which information contained within a gene is converted into the structures (e.g., a protein marker, such as PD-L1) present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). “Elevated expression,” “elevated expression levels,” or “elevated levels” refers to an increased expression or increased levels of a substance within a sample relative to a control sample, such as an individual or individuals who are not suffering from the disease or disorder (e.g., cancer) or an internal control. In some aspects, the elevated expression of a substance (e.g., a protein marker, such as PD-L1) in a sample refers to an increase in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g., FACS). “Reduced expression,” “reduced expression levels,” or “reduced levels” refers to a decrease expression or decreased levels of a substance (e.g., a protein marker) in an individual relative to a control, such as an individual or individuals who are not suffering from the disease or disorder (e.g., cancer) or an internal control. In some aspects, reduced expression is little or no expression. In some aspects, the reduced expression of a substance (e.g., a protein marker) in a sample refers to a decrease in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g, FACS).

As used herein, the term “antibody” refers to a whole antibody comprising two light chain polypeptides and two heavy chain polypeptides. Whole antibodies include different antibody isotypes including IgM, IgG, IgA, IgD, and IgE antibodies. The term “antibody” includes a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

As used herein, the term “antibody fragment”, “antigen-binding fragment”, “antigen-binding portion”, “antigen-binding protein” or similar terms refer to a fragment/portion of an antibody that retains the ability to bind to a target antigen (e.g., CCR8) and inhibit the activity of the target antigen. Such fragments include, e.g., a single chain antibody, a single chain Fv fragment (scFv), an Fd fragment, an Fab fragment, an Fab′ fragment, or an F(ab′)2 fragment. An scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein.

As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations). Affinity matured” (or “affinity matured antibody”): as used herein, refers to an antibody with one or more alterations in one or more CDRs thereof which result an improvement in the affinity of the antibody for antigen, compared to a parent antibody which does not possess those alteration(s). In some embodiments, affinity matured antibodies will have nanomolar or even picomolar affinities for a target antigen. Affinity matured antibodies may be produced by any of a variety of procedures known in the art.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc., In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Amino acid: in its broadest sense, as used herein, refers to any compound and/or substance that can be incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

As used herein, “cancer antigen” or “tumor antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

As used herein, the term “cancer-specific immune response” refers to the immune response induced by the presence of tumors, cancer cells, or cancer antigens. In certain aspects, the response includes the proliferation of cancer antigen specific lymphocytes. In certain aspects, the response includes expression and upregulation of antibodies and T-cell receptors and the formation and release of lymphokines, chemokines, and cytokines. Both innate and acquired immune systems interact to initiate antigenic responses against the tumors, cancer cells, or cancer antigens. In certain aspects, the cancer-specific immune response is a T cell response.

As used herein, the term “CCR8” or “C-C chemokine receptor type 8” refers to a G-protein coupled receptor.

As used herein, the term “combination therapy” refers to those situations in which a subject is simultaneously exposed to two or more therapeutic regimens (e.g., two or more therapeutic agents).

As used herein, “CDR” refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed invention.

As used herein the term “compete” when used in the context of antigen-binding proteins (e.g., immunoglobulins, antibodies, or antigen-binding fragments thereof) that compete for binding to the same epitope, refers to a interaction between antigen-binding proteins as determined by an assay (e.g., a competitive binding assay; a cross-blocking assay), wherein a test antigen-binding protein (e.g., a test antibody) inhibits (e.g., reduces or blocks) specific binding of a reference antigen-binding protein (e.g., a reference antibody) to a common antigen (e.g., CCR8 or a fragment thereof).

As used herein, the term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

As used herein, “bispecific antibody” refers to a bispecific binding agent in which at least one, and typically both, of the binding moieties is or comprises an antibody component. A variety of different bi-specific antibody structures are known in the art. In some embodiments, each binding moiety in a bispecific antibody that is or comprises an antibody component includes VH and/or VL regions; in some such embodiments, the VH and/or VL regions are those found in a particular monoclonal antibody. In some embodiments, where the bispecific antibody contains two antibody component-binding moieties, each includes VH and/or VL regions from different monoclonal antibodies. In some embodiments, where the bispecific antibody contains two antibody component binding moieties, wherein one of the two antibody component binding moieties includes an immunoglobulin molecule having VH and/or VL regions that contain CDRs from a first monoclonal antibody, and one of the two antibody component binding moieties includes an antibody fragment (e.g., Fab, F(ab′), F(ab′)2, Fd, Fv, dAB, scFv, etc.) having VH and/or VL regions that contain CDRs from a second monoclonal antibody.

As used herein, the term “human antibody” includes antibodies having variable and constant regions (if present) of human germline immunoglobulin sequences.

As used herein, the term “humanized” refers to an antibody in which some, most or all of the amino acids outside the CDR domains of a non-human antibody are replaced with corresponding amino acids derived from human immunoglobulins. In some embodiments of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

As used herein, the term a “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

As used herein, “antigen” refers to an agent that elicits an immune response; and/or (ii) an agent that binds to a T cell receptor (e.g., when presented by an MHC molecule) or to an antibody.

The terms “inducing an immune response” and “enhancing an immune response” are used interchangeably and refer to the stimulation of an immune response (i.e., either passive or adaptive) to a particular antigen. The terms “induce” as used with respect to inducing CDC or ADCC refer to the stimulation of particular direct cell killing mechanisms.

As used herein, the term “immunogenic cell death” (alternatively known as “immunogenic apoptosis” refers to a cell death modality associated with the activation of one or more signaling pathways that induces the pre-mortem expression and emission of damaged-associated molecular pattern (DAMPs) molecules (e.g., adenosine triphosphate, ATP) from the tumor cell, resulting in the increase of immunogenicity of the tumor cell and the death of the tumor cell in an immunogenic manner (e.g., by phagocytosis). As used herein, the term “immunogenic cell death-inducing agent” refers to a chemical, biological, or pharmacological agent that induces an immunogenic cell death process, pathway, or modality.

The term “in vivo” refers to processes that occur in a living organism.

As used herein the term “Kd” refers to the equilibrium dissociation constant of a binding reaction between an antibody and an antigen. The value of Kd is a numeric representation of the ratio of the antibody off-rate constant (kd) to the antibody on-rate constant (ka). The value of Kd is inversely related to the binding affinity of an antibody to an antigen. The smaller the Kd value the greater the affinity of the antibody for its antigen. Affinity is the strength of binding of a single molecule to its ligand and is typically measured and reported by the equilibrium dissociation constant (Kd), which is used to evaluate and rank order strengths of bimolecular interactions.

As used herein, the term “kd” (alternatively “koff) is intended to refer to the off-rate constant for the dissociation of an antibody from an antibody/antigen complex. The value of kd is a numeric representation of the fraction of complexes that decay or dissociate per second, and is expressed in units S⁻¹.

As used herein, the term “ka” (alternatively “kon”) is intended to refer to the on-rate constant for the association of an antibody with an antigen. The value of ka is a numeric representation of the number of antibody/antigen complexes formed per second in a 1 molar (1M) solution of antibody and antigen, and is expressed in units M⁻¹·S⁻¹.

As used herein, the term “monoclonal antibody” refers to an antibody that displays a single binding specificity and affinity for a particular epitope. Accordingly, the term “human monoclonal antibody” refers to an antibody which displays a single binding specificity and which has variable and optional constant regions derived from human germline immunoglobulin sequences. In some aspects, human monoclonal antibodies are produced by a hybridoma that includes a B cell obtained from a transgenic non-human animal, e.g., a transgenic mouse, having a genome comprising a human heavy chain transgene and a light chain transgene fused to an immortalized cell.

As used herein, the term “natural killer (NK) cell” refers to a type of cytotoxic lymphocyte. These are large, usually granular, non-T, non-B lymphocytes, which kill certain tumor cells and play an important role in innate immunity to viruses and other intracellular pathogens, as well as in antibody-dependent cell-mediated cytotoxicity (ADCC).

As used herein, the term “antibody-dependent cellular cytotoxicity” or “ADCC” refers to a phenomenon in which target cells bound by antibody are killed by immune effector cells. Without wishing to be bound by any particular theory, we observe that ADCC is typically understood to involve Fc receptor (FcR)-bearing effector cells can recognizing and subsequently killing antibody-coated target cells (e.g., cells that express on their surface specific antigens to which an antibody is bound). Effector cells that mediate ADCC can include immune cells, including but not limited to one or more of natural killer (NK) cells, macrophage, neutrophils, eosinophils.

As used herein, the term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof and complementary sequences and as well as the sequence explicitly indicated. Specifically, degenerate codon substitutions can be achieved by generating sequences in which the third position of one or more selected (or all) codons is substituted with mixed-base and/or deoxyinosine residues. For arginine and leucine, modifications at the second base can also be conservative. The term nucleic acid is used interchangeably with gene, cDNA, and mRNA encoded by a gene.

Polynucleotides used herein can be composed of any polyribonucleotide or polydeoxyribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the term “patient” includes human and other mammalian subjects that receive either prophylactic or therapeutic treatment.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt.

As used herein, the terms “polypeptide,” “peptide,” and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “preventing” when used in relation to a condition, refers to administration of a composition that reduces the frequency of, or delays the onset of, symptoms of a medical condition in a subject relative to a subject that does not receive the composition.

As used herein, the terms “specific binding,” “selective binding,” “selectively binds,” and “specifically binds,” refer to antibody binding to an epitope on a predetermined antigen.

As used herein, “epitope” includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

As used herein, the term “T cell-mediated response” refers to any response mediated by T cells, including, but not limited to, effector T cells (e.g., CD8+ cells) and helper T cells (e.g., CD4+ cells). T cell mediated responses include, for example, T cell cytotoxicity and proliferation.

As used herein, the term “regulatory T cell,” “T regulatory cell,” Treg,” or “Treg”, used interchangeably herein, refers to a subpopulation of T cells that modulate the immune system, maintain tolerance to self-antigens, and prevent autoimmune disease. Tregs are immunosuppressive and generally suppress or downregulate induction and proliferation of effector T cells. Tregs are known to direct effector T cell lysis, support tolerogenic dendritic cell formation, support M2 macrophage formation, produce immunosuppressive metabolites and cytokines, serve as an IL-2 sink, and to promote neovasculature formation. Though there are many types of Tregs, many Tregs express CD4 and FOXP3, with FOXP3 serving as a marker for Tregs in many cases.

As used herein, the terms “therapeutically effective amount” or “therapeutically effective dose,” or similar terms used herein are intended to mean an amount of an agent that will elicit the desired biological or medical response (e.g., an improvement in one or more symptoms of a cancer).

The terms “treat,” “treating,” and “treatment,” as used herein, refer to therapeutic or preventative measures described herein. The methods of “treatment” employ administration to a subject, in need of such treatment, a human antibody of the present disclosure, for example, a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

As used herein, the term “tumor microenvironment” (alternatively “cancer microenvironment;” abbreviated TME) refers to the cellular environment or milieu in which the tumor or neoplasm exists, including surrounding blood vessels as well as non-cancerous cells including, but not limited to, immune cells, fibroblasts, bone marrow-derived inflammatory cells, and lymphocytes. Signaling molecules and the extracellular matrix also comprise the TME. The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of tumor cells.

As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

As used herein, the term “solid tumor” refers to an abnormal mass of tissue that usually does not contain cysts or liquid areas. In some embodiments, a solid tumor may be benign; in some embodiments, a solid tumor may be malignant. Those skilled in the art will appreciate that different types of solid tumors are typically named for the type of cells that form them. Examples of solid tumors are carcinomas, lymphomas, and sarcomas.

Certain aspects of the present disclosure are directed to an anti-CCR8 antibody or antigen-binding portion thereof comprising a heavy chain CDR1 having any one of the amino acid sequences set forth in SEQ ID NOs: 1-2, 56-60, 62, a heavy chain CDR2 having the amino acid sequence set forth in SEQ ID NO: 3, 63-67, 69-73, a heavy chain CDR3 having any one of the amino acid sequences set forth in SEQ ID NOs: 4-5, 74-78, 80-85, a light chain CDR1 having any one of the amino acid sequences set forth in SEQ ID NOs: 6-11, 86-90, 92-93, a light chain CDR2 having the amino acid sequence set forth in SEQ ID NO: 12, 94-98, 100-102, and a light chain CDR3 having any one of the amino acid sequences set forth in SEQ ID NOs: 13-14, 103-107, 109-113.

In some aspects, in the anti-CCR8 antibody or antigen-binding portion thereof:
1) the HCDR3 has the amino acid sequence set forth in SEQ ID NO: 4, 74-78, 80-85; and
2) the LCDR1 has any one of the amino acid sequences set forth in SEQ ID NOs: 7, 10-11, 86-90, 92-93.

In some aspects, in the anti-CCR8 antibody or antigen-binding portion thereof:
1) the LCDR1 has any one of the amino acid sequences set forth in SEQ ID NOs: 10-11, 86-90, 92-93; and
2) the LCDR3 has any one of the amino acid sequences set forth in SEQ ID NO: 14, 103-107, 109-113.

In some aspects, in the anti-CCR8 antibody or antigen-binding portion thereof, the LCDR1 has the amino acid sequence set forth in SEQ ID NO: 10, 86-90, 92-93.

In some aspects, in the anti-CCR8 antibody or antigen-binding portion thereof:
1) the HCDR1 has the amino acid sequence set forth in SEQ ID NO: 1, the HCDR2 has the amino acid sequence set forth in SEQ ID NO: 3, the HCDR3 has the amino acid sequence set forth in SEQ ID NO: 4, the LCDRI has the amino acid sequence set forth in SEQ ID NO: 6, the LCDR2 has the amino acid sequence set forth in SEQ ID NO: 12 and the LCDR3 has the amino acid sequence set forth in SEQ ID NO: 13;
2) the HCDRI has the amino acid sequence set forth in SEQ ID NO: 2, the HCDR2 has the amino acid sequence set forth in SEQ ID NO: 3, the HCDR3 has the amino acid sequence set forth in SEQ ID NO: 5, the LCDRI has the amino acid sequence set forth in SEQ ID NO: 11, the LCDR2 has the amino acid sequence set forth in SEQ ID NO: 12 and the LCDR3 has the amino acid sequence set forth in SEQ ID NO: 14;
3) the HCDR1 has the amino acid sequence set forth in SEQ ID NO: 1, the HCDR2 has the amino acid sequence set forth in SEQ ID NO: 3, the HCDR3 has the amino acid sequence set forth in SEQ ID NO: 4, the LCDRI has the amino acid sequence set forth in SEQ ID NO: 7, the LCDR2 has the amino acid sequence set forth in SEQ ID NO: 12 and the LCDR3 has the amino acid sequence set forth in SEQ ID NO: 13;
4) the HCDR1 has the amino acid sequence set forth in SEQ ID NO: 2, the HCDR2 has the amino acid sequence set forth in SEQ ID NO: 3, the HCDR3 has the amino acid sequence set forth in SEQ ID NO: 5, the LCDR1 has the amino acid sequence set forth in SEQ ID NO: 8, the LCDR2 has the amino acid sequence set forth in SEQ ID NO: 12 and the LCDR3 has the amino acid sequence set forth in SEQ ID NO: 14;
5) the HCDR1 has the amino acid sequence set forth in SEQ ID NO: 2, the HCDR2 has the amino acid sequence set forth in SEQ ID NO: 3, the HCDR3 has the amino acid sequence set forth in SEQ ID NO: 5, the LCDR1 has the amino acid sequence set forth in SEQ ID NO: 9, the LCDR2 has the amino acid sequence set forth in SEQ ID NO: 12 and the LCDR3 has the amino acid sequence set forth in SEQ ID NO: 14;
6) the HCDR1 has the amino acid sequence set forth in SEQ ID NO: 2, the HCDR2 has the amino acid sequence set forth in SEQ ID NO: 3, the HCDR3 has the amino acid sequence set forth in SEQ ID NO: 5, the LCDR1 has the amino acid sequence set forth in SEQ ID NO: 10, the LCDR2 has the amino acid sequence set forth in SEQ ID NO: 12 and the LCDR3 has the amino acid sequence set forth in SEQ ID NO: 14;
7) the HCDR1 has the amino acid sequence set forth in SEQ ID NO: 1, the HCDR2 has the amino acid sequence set forth in SEQ ID NO: 3, the HCDR3 has the amino acid sequence set forth in SEQ ID NO: 4, the LCDR1 has the amino acid sequence set forth in SEQ ID NO: 11, the LCDR2 has the amino acid sequence set forth in SEQ ID NO: 12 and the LCDR3 has the amino acid sequence set forth in SEQ ID NO: 14;
8) the HCDR1 has the amino acid sequence set forth in SEQ ID NO: 1, the HCDR2 has the amino acid sequence set forth in SEQ ID NO: 3, the HCDR3 has the amino acid sequence set forth in SEQ ID NO: 4, the LCDRI has the amino acid sequence set forth in SEQ ID NO: 10, the LCDR2 has the amino acid sequence set forth in SEQ ID NO: 12 and the LCDR3 has the amino acid sequence set forth in SEQ ID NO: 14;
9) the heavy chain CDR1 has the amino acid sequence set forth in SEQ ID NO: 56 the heavy chain CDR2 has the amino acid sequence set forth in SEQ ID NO: 63, the heavy chain CDR3 has the amino acid sequence set forth in SEQ ID NO: 74, the light chain CDR1 has the amino acid sequence set forth in SEQ ID NO: 86, the light chain CDR2 has the amino acid sequence set forth in SEQ ID NO: 94 and the light chain CDR3 has the amino acid sequence set forth in SEQ ID NO: 103;
10) the heavy chain CDR1 has the amino acid sequence set forth in SEQ ID NO: 57, the heavy chain CDR2 has the amino acid sequence set forth in SEQ ID NO: 64, the heavy chain CDR3 has the amino acid sequence set forth in SEQ ID NO: 75, the light chain CDR1 has the amino acid sequence set forth in SEQ ID NO: 87, the light chain CDR2 has the amino acid sequence set forth in SEQ ID NO: 95 and the light chain CDR3 has the amino acid sequence set forth in SEQ ID NO: 104;
11) the heavy chain CDR1 has the amino acid sequence set forth in SEQ ID NO: 56, the heavy chain CDR2 has the amino acid sequence set forth in SEQ ID NO: 63, the heavy chain CDR3 has the amino acid sequence set forth in SEQ ID NO: 74, the light chain CDR1 has the amino acid sequence set forth in SEQ ID NO: 86, the light chain CDR2 has the amino acid sequence set forth in SEQ ID NO: 94 and the light chain CDR3 has the amino acid sequence set forth in SEQ ID NO: 103;
12) the heavy chain CDR1 has the amino acid sequence set forth in SEQ ID NO: 57, the heavy chain CDR2 has the amino acid sequence set forth in SEQ ID NO: 64, the heavy chain CDR3 has the amino acid sequence set forth in SEQ ID NO: 75, the light chain CDR1 has the amino acid sequence set forth in SEQ ID NO: 87, the light chain CDR2 has the amino acid sequence set forth in SEQ ID NO: 95 and the light chain CDR3 has the amino acid sequence set forth in SEQ ID NO: 104;
13) the heavy chain CDR1 has the amino acid sequence set forth in SEQ ID NO: 58, the heavy chain CDR2 has the amino acid sequence set forth in SEQ ID NO: 65, the heavy chain CDR3 has the amino acid sequence set forth in SEQ ID NO: 76, the light chain CDR1 has the amino acid sequence set forth in SEQ ID NO: 88, the light chain CDR2 has the amino acid sequence set forth in SEQ ID NO: 96 and the light chain CDR3 has the amino acid sequence set forth in SEQ ID NO: 105;
14) the heavy chain CDR1 has the amino acid sequence set forth in SEQ ID NO: 59, the heavy chain CDR2 has the amino acid sequence set forth in SEQ ID NO: 66, the heavy chain CDR3 has the amino acid sequence set forth in SEQ ID NO: 77, the light chain CDR1 has the amino acid sequence set forth in SEQ ID NO: 89, the light chain CDR2 has the amino acid sequence set forth in SEQ ID NO: 97 and the light chain CDR3 has the amino acid sequence set forth in SEQ ID NO: 106;
15) the heavy chain CDR1 has the amino acid sequence set forth in SEQ ID NO: 60, the heavy chain CDR2 has the amino acid sequence set forth in SEQ ID NO: 67, the heavy chain CDR3 has the amino acid sequence set forth in SEQ ID NO: 78, the light chain CDRI has the amino acid sequence set forth in SEQ ID NO: 90, the light chain CDR2 has the amino acid sequence set forth in SEQ ID NO: 98 and the light chain CDR3 has the amino acid sequence set forth in SEQ ID NO: 107;
16) the heavy chain CDR1 has the amino acid sequence set forth in SEQ ID NO: 58, the heavy chain CDR2 has the amino acid sequence set forth in SEQ ID NO: 69, the heavy chain CDR3 has the amino acid sequence set forth in SEQ ID NO: 80, the light chain CDR1 has the amino acid sequence set forth in SEQ ID NO: 88, the light chain CDR2 has the amino acid sequence set forth in SEQ ID NO: 100 and the light chain CDR3 has the amino acid sequence set forth in SEQ ID NO: 109;
17) the heavy chain CDR1 has the amino acid sequence set forth in SEQ ID NO: 58, the heavy chain CDR2 has the amino acid sequence set forth in SEQ ID NO: 70, the heavy chain CDR3 has the amino acid sequence set forth in SEQ ID NO: 81, the light chain CDR1 has the amino acid sequence set forth in SEQ ID NO: 88, the light chain CDR2 has the amino acid sequence set forth in SEQ ID NO: 100 and the light chain CDR3 has the amino acid sequence set forth in SEQ ID NO: 109;
18) the heavy chain CDR1 has the amino acid sequence set forth in SEQ ID NO: 58, the heavy chain CDR2 has the amino acid sequence set forth in SEQ ID NO: 65, the heavy chain CDR3 has the amino acid sequence set forth in SEQ ID NO: 82, the light chain CDR1 has the amino acid sequence set forth in SEQ ID NO: 88, the light chain CDR2 has the amino acid sequence set forth in SEQ ID NO: 101 and the light chain CDR3 has the amino acid sequence set forth in SEQ ID NO: 110;
19) the heavy chain CDR1 has the amino acid sequence set forth in SEQ ID NO: 62, the heavy chain CDR2 has the amino acid sequence set forth in SEQ ID NO: 71, the heavy chain CDR3 has the amino acid sequence set forth in SEQ ID NO: 83, the light chain CDR1 has the amino acid sequence set forth in SEQ ID NO: 92, the light chain CDR2 has the amino acid sequence set forth in SEQ ID NO: 102 and the light chain CDR3 has the amino acid sequence set forth in SEQ ID NO: 111;
20) the heavy chain CDR1 has the amino acid sequence set forth in SEQ ID NO: 58, the heavy chain CDR2 has the amino acid sequence set forth in SEQ ID NO: 72, the heavy chain CDR3 has the amino acid sequence set forth in SEQ ID NO: 84, the light chain CDRI has the amino acid sequence set forth in SEQ ID NO: 93, the light chain CDR2 has the amino acid sequence set forth in SEQ ID NO: 100 and the light chain CDR3 has the amino acid sequence set forth in SEQ ID NO: 112; or
21) the heavy chain CDR1 has the amino acid sequence set forth in SEQ ID NO: 58, the heavy chain CDR2 has the amino acid sequence set forth in SEQ ID NO: 73, the heavy chain CDR3 has the amino acid sequence set forth in SEQ ID NO: 85, the light chain CDR1 has the amino acid sequence set forth in SEQ ID NO: 88, the light chain CDR2 has the amino acid sequence set forth in SEQ ID NO: 100 and the light chain CDR3 has the amino acid sequence set forth in SEQ ID NO: 113.

Certain aspects of the present disclosure are directed to an anti-CCR8 antibody or antigen-binding portion thereof comprising a variable region of heavy chain (VH), wherein the VH has any one of the amino acid sequences set forth in SEQ ID NOs: 15-20, 114-120, 122-128.

In certain aspects, the anti-CCR8 antibody or antigen-binding protein comprises a variable region of heavy chain (VH), wherein the VH has any one of the amino acid sequences set forth in SEQ ID NOs: 16-20, 114-120, 122-128.

In certain aspects, the anti-CCR8 antibody or antigen-binding protein comprises a variable region of heavy chain (VH), wherein the VH has the amino acid sequence set forth in SEQ ID NO: 18, 114-120, 122-128.

Certain aspects of the present disclosure are directed to an anti-CCR8 antibody or antigen-binding portion thereof comprising a variable region of light chain (VL), wherein the VL has any one of the amino acid sequences set forth in SEQ ID NOs: 21-28, 129-135, 137-143.

In certain aspects, the anti-CCR8 antibody or antigen-binding protein comprises a variable region of light chain (VL), wherein the VL has any one of the amino acid sequences set forth in SEQ ID NOs: 22-28, 129-135, 137-143.

In certain aspects, the anti-CCR8 antibody or antigen-binding protein comprises a variable region of light chain (VL), wherein the VL has any one of the amino acid sequences set forth in SEQ ID NOs: 24, 25, 28, 129-135, 137-143.

In certain aspects, the anti-CCR8 antibody or antigen-binding protein comprises a variable region of light chain (VL), wherein the VL has any one of the amino acid sequences set forth in SEQ ID NOs: 25, 28, 129-135, 137-143.

In certain aspects, the anti-CCR8 antibody or antigen-binding protein comprises a variable region of light chain (VL), wherein the VL has the amino acid sequence set forth in SEQ ID NO: 28, 129-135, 137-143.

In certain aspects, the anti-CCR8 antibody or antigen-binding protein comprises a variable region of heavy chain (VH) and a variable region of light chain (VL), wherein:
1) the VH has the amino acid sequence set forth in SEQ ID NO: 15 and the VL has the amino acid sequence set forth in SEQ ID NO: 21;
2) the VH has the amino acid sequence set forth in SEQ ID NO: 16 and the VL has the amino acid sequence set forth in SEQ ID NO: 22;
3) the VH has the amino acid sequence set forth in SEQ ID NO: 17 and the VL has the amino acid sequence set forth in SEQ ID NO: 23;
4) the VH has the amino acid sequence set forth in SEQ ID NO: 18 and the VL has the amino acid sequence set forth in SEQ ID NO: 23;
5) the VH has the amino acid sequence set forth in SEQ ID NO: 19 and the VL has the amino acid sequence set forth in SEQ ID NO: 23;
6) the VH has the amino acid sequence set forth in SEQ ID NO: 18 and the VL has the amino acid sequence set forth in SEQ ID NO: 24;
7) the VH has the amino acid sequence set forth in SEQ ID NO: 19 and the VL has the amino acid sequence set forth in SEQ ID NO: 24;
8) the VH has the amino acid sequence set forth in SEQ ID NO: 20 and the VL has the amino acid sequence set forth in SEQ ID NO: 25;
9) the VH has the amino acid sequence set forth in SEQ ID NO: 20 and the VL has the amino acid sequence set forth in SEQ ID NO: 26;
10) the VH has the amino acid sequence set forth in SEQ ID NO: 20 and the VL has the amino acid sequence set forth in SEQ ID NO: 27;
11) the VH has the amino acid sequence set forth in SEQ ID NO: 20 and the VL has the amino acid sequence set forth in SEQ ID NO: 28;
12) the VH has the amino acid sequence set forth in SEQ ID NO: 18 and the VL has the amino acid sequence set forth in SEQ ID NO: 25;
13) the VH has the amino acid sequence set forth in SEQ ID NO: 18 and the VL has the amino acid sequence set forth in SEQ ID NO: 28;
14) the variable region of heavy chain has the amino acid sequence set forth in SEQ ID NO: 114 and the variable region of light chain has the amino acid sequence set forth in SEQ ID NO: 129;
15) the variable region of heavy chain has the amino acid sequence set forth in SEQ ID NO: 115 and the variable region of light chain has the amino acid sequence set forth in SEQ ID NO: 130;
16) the variable region of heavy chain has the amino acid sequence set forth in SEQ ID NO: 116 and the variable region of light chain has the amino acid sequence set forth in SEQ ID NO: 131;
17) the variable region of heavy chain has the amino acid sequence set forth in SEQ ID NO: 117 and the variable region of light chain has the amino acid sequence set forth in SEQ ID NO: 132;
18) the variable region of heavy chain has the amino acid sequence set forth in SEQ ID NO: 118 and the variable region of light chain has the amino acid sequence set forth in SEQ ID NO: 133;
19) the variable region of heavy chain has the amino acid sequence set forth in SEQ ID NO: 119 and the variable region of light chain has the amino acid sequence set forth in SEQ ID NO: 134;
20) the variable region of heavy chain has the amino acid sequence set forth in SEQ ID NO: 120 and the variable region of light chain has the amino acid sequence set forth in SEQ ID NO: 135;
21) the variable region of heavy chain has the amino acid sequence set forth in SEQ ID NO: 122 and the variable region of light chain has the amino acid sequence set forth in SEQ ID NO: 137;
22) the variable region of heavy chain has the amino acid sequence set forth in SEQ ID NO: 123 and the variable region of light chain has the amino acid sequence set forth in SEQ ID NO: 138;
23) the variable region of heavy chain has the amino acid sequence set forth in SEQ ID NO: 124 and the variable region of light chain has the amino acid sequence set forth in SEQ ID NO: 139;
24) the variable region of heavy chain has the amino acid sequence set forth in SEQ ID NO: 125 and the variable region of light chain has the amino acid sequence set forth in SEQ ID NO: 140;
25) the variable region of heavy chain has the amino acid sequence set forth in SEQ ID NO: 126 and the variable region of light chain has the amino acid sequence set forth in SEQ ID NO: 141;
26) the variable region of heavy chain has the amino acid sequence set forth in SEQ ID NO: 127 and the variable region of light chain has the amino acid sequence set forth in SEQ ID NO: 142; or
27) the variable region of heavy chain has the amino acid sequence set forth in SEQ ID NO: 127 and the variable region of light chain has the amino acid sequence set forth in SEQ ID NO: 143.

Certain aspects of the present disclosure are directed to an anti-CCR8 antibody or antigen-binding portion thereof comprising a constant region of heavy chain (CH), wherein the CH has any one of the amino acid sequences set forth in SEQ ID NOs: 29-30.

In certain aspects, the anti-CCR8 antibody or antigen-binding protein comprises a constant region of heavy chain (CH), wherein the CH has the amino acid sequence set forth in SEQ ID NO: 30.

In certain aspects, the anti-CCR8 antibody or antigen-binding protein comprises a constant region of light chain (CL), wherein the CL has the amino acid sequence set forth in SEQ ID NO: 31.

In certain aspects, the anti-CCR8 antibody or antigen-binding protein comprises a constant region of heavy chain (CH) and a constant region of light chain (CL), wherein:
1) the CH has the amino acid sequence set forth in SEQ ID NO: 29 and the CL has the amino acid sequence set forth in SEQ ID NO: 31;
2) the CH has the amino acid sequence set forth in SEQ ID NO: 30 and the CL has the amino acid sequence set forth in SEQ ID NO: 31;

Certain aspects of the present disclosure are directed to an anti-CCR8 antibody or antigen-binding portion thereof comprising a heavy chain, wherein the heavy chain has any one of the amino acid sequences set forth in SEQ ID NOs: 32-39, 144-150, 152-158.

In certain aspects, the anti-CCR8 antibody or antigen-binding protein comprises a heavy chain, wherein the heavy chain has any one of the amino acid sequences set forth in SEQ ID NOs: 34-39, 144-150, 152-158.

In certain aspects, the anti-CCR8 antibody or antigen-binding protein comprises a heavy chain, wherein the heavy chain has the amino acid sequence set forth in SEQ ID NO: 37, 144-150, 152-158.

Certain aspects of the present disclosure are directed to an anti-CCR8 antibody or antigen-binding portion thereof comprising a light chain, wherein the light chain has any one of the amino acid sequences set forth in SEQ ID NOs: 40-47, 159-165, 167-173.

In certain aspects, the anti-CCR8 antibody or antigen-binding protein comprises a light chain, wherein the light chain has any one of the amino acid sequences set forth in SEQ ID NOs: 41-47, 159-165, 167-173.

In certain aspects, the anti-CCR8 antibody or antigen-binding protein comprises a light chain, wherein the light chain has any one of the amino acid sequences set forth in SEQ ID NOs: 43, 44 and 47, 159-165, 167-173.

In certain aspects, the anti-CCR8 antibody or antigen-binding protein comprises a light chain, wherein the light chain has any one of the amino acid sequences set forth in SEQ ID NOs: 44 and 47, 159-165, 167-173.

In certain aspects, the anti-CCR8 antibody or antigen-binding protein comprises a light chain, wherein the light chain has the amino acid sequence set forth in SEQ ID NO: 47, 159-165, 167-173.

In certain aspects, the anti-CCR8 antibody or antigen-binding protein comprises a heavy chain and a light chain, wherein:
1) the heavy chain has the amino acid sequence set forth in SEQ ID NO: 32 and the light chain has the amino acid sequence set forth in SEQ ID NO: 40;
2) the heavy chain has the amino acid sequence set forth in SEQ ID NO: 33 and the light chain has the amino acid sequence set forth in SEQ ID NO: 41;
3) the heavy chain has the amino acid sequence set forth in SEQ ID NO: 34 and the light chain has the amino acid sequence set forth in SEQ ID NO: 42;
4) the heavy chain has the amino acid sequence set forth in SEQ ID NO: 35 and the light chain has the amino acid sequence set forth in SEQ ID NO: 42;
5) the heavy chain has the amino acid sequence set forth in SEQ ID NO: 36 and the light chain has the amino acid sequence set forth in SEQ ID NO: 42;
6) the heavy chain has the amino acid sequence set forth in SEQ ID NO: 35 and the light chain has the amino acid sequence set forth in SEQ ID NO: 43;
7) the heavy chain has the amino acid sequence set forth in SEQ ID NO: 36 and the light chain has the amino acid sequence set forth in SEQ ID NO: 43;
8) the heavy chain has the amino acid sequence set forth in SEQ ID NO: 38 and the light chain has the amino acid sequence set forth in SEQ ID NO: 41;
9) the heavy chain has the amino acid sequence set forth in SEQ ID NO: 39 and the light chain has the amino acid sequence set forth in SEQ ID NO: 44;
10) the heavy chain has the amino acid sequence set forth in SEQ ID NO: 39 and the light chain has the amino acid sequence set forth in SEQ ID NO: 45;
11) the heavy chain has the amino acid sequence set forth in SEQ ID NO: 39 and the light chain has the amino acid sequence set forth in SEQ ID NO: 46;
12) the heavy chain has the amino acid sequence set forth in SEQ ID NO: 39 and the light chain has the amino acid sequence set forth in SEQ ID NO: 47;
13) the heavy chain has the amino acid sequence set forth in SEQ ID NO: 37 and the light chain has the amino acid sequence set forth in SEQ ID NO: 43;
14) the heavy chain has the amino acid sequence set forth in SEQ ID NO: 37 and the light chain has the amino acid sequence set forth in SEQ ID NO: 44;
15) the heavy chain has the amino acid sequence set forth in SEQ ID NO: 37 and the light chain has the amino acid sequence set forth in SEQ ID NO: 47;
16) the heavy chain has the amino acid sequence set forth in SEQ ID NO: 144 and the light chain has the amino acid sequence set forth in SEQ ID NO: 159;
17) the heavy chain has the amino acid sequence set forth in SEQ ID NO: 145 and the light chain has the amino acid sequence set forth in SEQ ID NO: 160;
18) the heavy chain has the amino acid sequence set forth in SEQ ID NO: 146 and the light chain has the amino acid sequence set forth in SEQ ID NO: 161;
19) the heavy chain has the amino acid sequence set forth in SEQ ID NO: 147 and the light chain has the amino acid sequence set forth in SEQ ID NO: 162;
20) the heavy chain has the amino acid sequence set forth in SEQ ID NO: 148 and the light chain has the amino acid sequence set forth in SEQ ID NO: 163;
21) the heavy chain has the amino acid sequence set forth in SEQ ID NO: 149 and the light chain has the amino acid sequence set forth in SEQ ID NO: 164;
22) the heavy chain has the amino acid sequence set forth in SEQ ID NO: 150 and the light chain has the amino acid sequence set forth in SEQ ID NO: 165;
23) the heavy chain has the amino acid sequence set forth in SEQ ID NO: 152 and the light chain has the amino acid sequence set forth in SEQ ID NO: 167;
24) the heavy chain has the amino acid sequence set forth in SEQ ID NO: 153 and the light chain has the amino acid sequence set forth in SEQ ID NO: 168;
25) the heavy chain has the amino acid sequence set forth in SEQ ID NO: 154 and the light chain has the amino acid sequence set forth in SEQ ID NO: 169;
26) the heavy chain has the amino acid sequence set forth in SEQ ID NO: 155 and the light chain has the amino acid sequence set forth in SEQ ID NO: 170;
27) the heavy chain has the amino acid sequence set forth in SEQ ID NO: 156 and the light chain has the amino acid sequence set forth in SEQ ID NO: 171;
28) the heavy chain has the amino acid sequence set forth in SEQ ID NO: 157 and the light chain has the amino acid sequence set forth in SEQ ID NO: 172; or
29) the heavy chain has the amino acid sequence set forth in SEQ ID NO: 158 and the light chain has the amino acid sequence set forth in SEQ ID NO: 173.

Certain aspects of the present disclosure are directed to an anti-CCR8 antibody or antigen-binding portion thereof that is afucosylated.

Certain aspects of the present disclosure are directed to an anti-CCR8 antibody or antigen-binding portion thereof, which induces antibody-dependent cellular cytotoxicity (ADCC) in a subject following administration of the anti-CCR8 antibody or antigen-binding portion thereof.

Certain aspects of the present disclosure are directed to an anti-CCR8 antibody or antigen-binding portion thereof, which is capable of inducing activation of NK cells.

In certain aspects, the present disclosure is directed to an anti-CCR8 antibody or antigen-binding portion thereof, which is capable of inducing NK cell mediated killing of tumor infiltrating Treg cells in a subject.

In certain aspects, the present disclosure is directed to an anti-CCR8 antibody or antigen-binding portion thereof, which induces internalization of CCR8 by tumor infiltrating Treg cells.

Certain aspects of the present disclosure are directed to an anti-CCR8 antibody or antigen-binding portion thereof, which is a human antibody, a humanized antibody, or a chimeric antibody or antigen-binding portion thereof.

Certain aspects of the present disclosure are directed to an anti-CCR8 antibody or antigen-binding portion thereof, which is an IgG1, IgG2, IgG3, or IgG4 isotype antibody or antigen binding fragment thereof.

Certain aspects of the present disclosure are directed to an anti-CCR8 antibody or antigen-binding portion thereof, which is a monoclonal antibody, a bispecific antibody, a bispecific T cell engager (BiTE), a multispecific antibody, a biparatopic antibody, an immunoconjugate, an antibody drug conjugate, a chimeric antigen receptor (CAR), a T cell receptor (TCR) or any combination thereof.

Certain aspects of the present disclosure are directed to an anti-CCR8 antibody or antigen-binding portion thereof, which targets the extracellular domain of human CCR8.

In certain aspects, the present disclosure is directed to an anti-CCR8 antibody or antigen-binding portion thereof, which binds cells expressing CCR8 on their surface.

In certain aspects, the present disclosure is directed to an anti-CCR8 antibody or antigen-binding portion thereof, which specifically binds to one or more amino acids within the N-terminal extracellular domain of CCR8.

In certain aspects, the present disclosure is directed to an anti-CCR8 antibody or antigen-binding portion thereof, which binds cells expressing CCR8 on their surface, wherein the cells are regulatory T cells (Tregs).

In certain aspects, the present disclosure is directed to an anti-CCR8 antibody or antigen-binding portion thereof, which binds cells expressing CCR8 on their surface, wherein the cells are tumor infiltrating Tregs.

Certain aspects of the present disclosure are directed to an anti-CCR8 antibody or antigen-binding portion thereof, which competes for binding with CCL1.

Certain aspects of the present disclosure are directed to a pharmaceutical composition comprising the anti-CCR8 antibody or antigen-binding portion thereof according to the present disclosure and a pharmaceutically acceptable carrier.

Certain aspects of the present disclosure are directed to a method of treating a disease or condition in a subject in need thereof, comprising administering to the subject the anti-CCR8 antibody or antigen-binding portion according to the present disclosure or the pharmaceutical composition according to the present disclosure.

In certain aspects, the disease or condition comprises tumor.

In certain aspects, the present disclosure is directed to a method of treating a tumor in a subject in need thereof, wherein the tumor comprises Kaposi's sarcoma, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chondrosarcoma, osteogenic sarcoma, osteosarcoma, chordoma, angiosarcoma, endotheliosarcoma, lymphangiosarcoma, lymphangio endothelial sarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma (HCC), hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system, and any combination thereof.

In certain aspects, the present disclosure is directed to a method of treating a tumor in a subject in need thereof, wherein the tumor is refractory or relapsed.

In certain aspects, the present disclosure is directed to a method of treating a tumor in a subject in need thereof, wherein the tumor is advanced, locally advanced, or metastatic.

In certain aspects, the present disclosure is directed to a method of treating a tumor in a subject in need thereof, which further comprises administering an additional anticancer agent and/or method.

Certain aspects of the present disclosure are directed to use of the anti-CCR8 antibody or antigen-binding portion thereof according to the present disclosure in the manufacture of a pharmaceutical for treating a disease or condition.

In certain aspects, the disease or condition comprises tumor.

In certain aspects, the present disclosure is directed to a method of treating a tumor in a subject in need thereof, wherein the tumor comprises Kaposi's sarcoma, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma (HCC), hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system, and any combination thereof.

In certain aspects, the present disclosure is directed to a method of treating a tumor in a subject in need thereof, wherein the tumor is refractory or relapsed.

In certain aspects, the present disclosure is directed to a method of treating a tumor in a subject in need thereof, wherein the tumor is advanced, locally advanced, or metastatic.

In certain aspects, the present disclosure is directed to a method of treating a tumor in a subject in need thereof, which further comprises administering an additional anticancer agent and/or method.

### EXAMPLES

### Example 1: Generation of Various Anti-CCR8 Antibodies

### 1.1 Hybridoma

To generate antibodies against CCR8, Sprague-Dawley rats were immunized with CCR8-overexpressing cells using various prime-boost strategies. To monitor immune responses, titrated serum was screened for binding to multiple CCR8-overexpressing cell lines by FACS, typically after 4-6 weeks of immunization. Animals with sufficient titers of anti-CCR8 IgG were selected for the final immunization.

For hybridoma generation, cells from lymphoid organs, such as spleens and lymph nodes were collected, isolated and fused with SP2/0 myeloma cells. The resulting cells were plated in flat-bottom 96 well plates in Medium E (StemCell Technologies) supplemented with HAT (Sigma H0262-10VL) to select for hybridomas. After one week of culture, hybridoma supernatants were collected and screened against cell lines overexpressing CCR8 by flow cytometry. Anti-CCR8 antibody-secreting hybridomas were subsequently subcloned and further screened to identify anti-CCR8 monoclonal hybridomas.

### 1.2 Generation of Expression Vectors

To obtain the sequences of the HC and LC variable domains, mRNA from the hybridomas were purified using Dynabeads mRNA Direct Micro Purification kit (ThermoFisher #61021) and the cDNAs were synthesized and amplified using template-switching oligos (Pinto et al., Anal. Biochem. 397, 2010). The variable domain fragments of the heavy and light chains were subsequently amplified, purified, and sequenced.

To generate the expression vectors, the variable regions of heavy and light chain DNA sequences were synthesized and subcloned in-frame with either the human IgG1 constant heavy chain with SDIE mutations or the human IgG1 kappa constant light chain pre-inserted into the pCI-vector (Promega #E1841).

### 1.3 Humanization of Antibodies

To humanize antibodies, the CDR residues of the light and heavy chains were grafted onto the human frameworks with high homology. To restore affinity, some mutations were designed by changing human framework residues to their murine counterparts at specific positions.

### 1.4 Production of Antibodies

Antibodies were produced by co-transfecting plasmids into Expi293F cells (ThermoFisher A14527). Specifically, 30 μg of total DNA was diluted into Opti-MEM medium (Life Technologies), and then incubated with Expifectamine 293 transfection reagent (Gibco #A14525) in the same medium for 20 min. The mixture was then added to 30 ml of Expi293F cells growing in suspension at a density of 2.5 million cells/ml at 37° C. and 8% CO2.

The cells were grown in suspension for about six days after transfection and then harvested by centrifugation at 7,000 rpm, 4° C. for 20 minutes. The supernatant was filtered through a 0.22 μm filter, and affinity purification was performed using protein A resins. The protein was eluted by elution buffer (1 M Glycine pH 3.0, 10% glycerol) and neutralized to pH 6.0 with 1M Tris pH 7.5. Size exclusion chromatography (SEC) was performed on AKTA to refine the product, and the antibody concentration was measured with NanoDrop at 280 nm. The antibodies were analyzed by electrophoresis on 4-20% Tris-glycine gels (Bio-Rad) under reducing (R) and non-reducing (NR) conditions.

The antibodies listed in Table 1 below were obtained by using 433H (BD #566379) or B1 (Biolegend #360602) as the parent antibody, through conventional experimental operations for antibody humanization well known in the art. These operations include, but are not limited to, change of IgG sub-class, re-humanization, change of signal peptide, amino acid point mutation, and/or a combination of several of these methods. For example, chimera antibodies (ChH2 and ChB1) were constructed by grafting the mouse antibody VL and VH regions of 433H or B1 onto human IgG1 constant regions.

Afucosylated antibodies were produced by transfecting heavy and light chain plasmids of the antibodies of interest into CHO cells deficient in mammalian a1,6-fucosyltransferase (FUT8) (Sino Biologics). After 7 days, the media was harvested, and the antibodies were purified using a protein A column. The purified antibodies were analyzed by SDS-PAGE and SEC-HPLC. As a result, three afucosylated antibodies were obtained: ChH2-AF, ABC024-H2G.AF, and ChB1-AF.

1.5 Analytical Test for mAbs (HPLC)

The purity and monomer content of the final purified proteins were determined by high-throughput analysis on HPLC. Size exclusion chromatography (SEC) was performed using an Advancebio SEC 300A 4.6×300 mm, 2.7 μm (Agilent PL1580-5301) on an Infinity 1260 II Bio-Inert Agilent HPLC system. Injections were made under isocratic elution conditions using a mobile phase of PBS and detected with absorbance at 280 nm. Quantification was based on the relative area of detected peaks.

A subject antibody can be substantially pure, e.g., at least about 80% to 85% pure, at least about 85% to 90% pure, at least about 90% to 95% pure, or 98% to 99%, or more, pure, e.g., free from contaminants such as cell debris, macromolecules other than a subject antibody, etc.

The full-length amino acid sequences of these antibodies are shown in Table 1 and Table 2.

TABLE 1
Amino acid sequences of the antibodies of the present disclosure

| ID | Antibody | Heavy chain | Light chain |
|---|---|---|---|
| 1 | ChH2 | SEQ ID NO: 32 | SEQ ID NO: 40 |
| 2 | ChB1-AF | SEQ ID NO: 33 | SEQ ID NO: 41 |
| 3 | H2A | SEQ ID NO: 34 | SEQ ID NO: 42 |
| 4 | H2B | SEQ ID NO: 35 | SEQ ID NO: 42 |
| 5 | H2C | SEQ ID NO: 36 | SEQ ID NO: 42 |
| 6 | H2G | SEQ ID NO: 35 | SEQ ID NO: 43 |
| 7 | H2I | SEQ ID NO: 36 | SEQ ID NO: 43 |
| 8 | ABC047-B1.IgG1.SDIE | SEQ ID NO: 38 | SEQ ID NO: 41 |
| 9 | ABC048-B1.hu11.IgG1.SDIE | SEQ ID NO: 39 | SEQ ID NO: 44 |
| 10 | ABC049-B1.hu12.IgG1.SDIE | SEQ ID NO: 39 | SEQ ID NO: 45 |
| 11 | ABC050-B1.hu13.IgG1.SDIE | SEQ ID NO: 39 | SEQ ID NO: 46 |
| 12 | ABC051-B1.hu14.IgG1.SDIE | SEQ ID NO: 39 | SEQ ID NO: 47 |
| 13 | ABC025-H2G.SDIE | SEQ ID NO: 37 | SEQ ID NO: 43 |
| 14 | ABC137-H2.hu12.IgG1.SDIE | SEQ ID NO: 37 | SEQ ID NO: 44 |
| 15 | ABC138-H2.hu13.IgG1.SDIE | SEQ ID NO: 37 | SEQ ID NO: 47 |
| 16 | ChH2-AF | SEQ ID NO: 32 | SEQ ID NO: 40 |
| 17 | ChB1-AF | SEQ ID NO: 33 | SEQ ID NO: 41 |
| 18 | ABC024-H2G.AF | SEQ ID NO: 35 | SEQ ID NO: 43 |
| 19 | ABC024-H2G | SEQ ID NO: 35 | SEQ ID NO: 43 |

TABLE 2
Amino acid sequences of the antibodies of the present disclosure

| ID | Antibody | Heavy chain | Light chain |
|---|---|---|---|
| 1 | ABC519 | SEQ ID NO: 144 | SEQ ID NO: 159 |
| 2 | ABC523 | SEQ ID NO: 145 | SEQ ID NO: 160 |
| 3 | ABC527 | SEQ ID NO: 146 | SEQ ID NO: 161 |
| 4 | ABC531 | SEQ ID NO: 147 | SEQ ID NO: 162 |
| 5 | ABC814 | SEQ ID NO: 148 | SEQ ID NO: 163 |
| 6 | ABC815 | SEQ ID NO: 149 | SEQ ID NO: 164 |
| 7 | ABC816 | SEQ ID NO: 150 | SEQ ID NO: 165 |
| 8 | ABC818 | SEQ ID NO: 152 | SEQ ID NO: 167 |
| 9 | ABC819 | SEQ ID NO: 153 | SEQ ID NO: 168 |
| 10 | ABC820 | SEQ ID NO: 154 | SEQ ID NO: 169 |
| 11 | ABC821 | SEQ ID NO: 155 | SEQ ID NO: 170 |
| 12 | ABC822 | SEQ ID NO: 156 | SEQ ID NO: 171 |
| 13 | ABC823 | SEQ ID NO: 157 | SEQ ID NO: 172 |
| 14 | ABC1183 | SEQ ID NO: 158 | SEQ ID NO: 173 |

### Example 2: Cellular Binding or Affinity of Anti-CCR8 Antibodies

### 2.1 Generating Stable Cell Lines Overexpressing CCR8

To generate cell lines for animal immunization and antibody characterization, cell lines with overexpressed CCR8 were developed. Nucleotide sequence (SEQ ID NO: 50) encoding Human CCR8 (Uniprot #P51685, SEQ ID NO:51) was cloned into pIREShygro3gGFP, which is modified from pIREShyg3 (Takara #631620) by fusing eGFP cDNA having the nucleotide sequence of SEQ ID No:48 to the 3′ Hygromycin B phosphotransferase gene in pIREShyg3 with a linker having the nucleotide sequence of SEQ ID No:49. The resulting plasmids were then transfected into HEK293T cells (ATCC CRL-3216) and C6 cells (ATCC CCL-107) using lipofectamine 3000 (ThermoFisher, L3000001). After three days, the cells were treated with hygromycin B (Millipore Sigma) for two weeks to select for stable cell lines with high CCR8 cell surface expression.

### 2.2 Cell-Based Binding Assays for Human CCR8 on Cell Lines

Binding specificity of the anti-CCR8 antibodies (mAbs) was tested by FACS using several cell lines. TALL-1 (DSMZ #ACC 521) and Hut78 cell lines (ATCC #TIB-161) were used, both of which harbor endogenous human CCR8 on the cell surface, along with HEK293T cells overexpressing CCR8. The binding titration of the anti-CCR8 antibodies to CCR8 transfectants was performed by serial dilution of antibodies. The diluted antibody (50 μl) in flow cytometry buffer (PBS, 0.5% BSA, 1 mM EDTA) was incubated with cells (50 μl) at a concentration of 2 million cells/ml on ice for 30 min. After two washes with flow cytometry buffer, bound antibody was detected with Alexa647-labeled F(ab′)2 fragment goat anti-human IgG (Jackson ImmunoResearch #709-606-149) (1:500 dilution) for 20 minutes on ice. The fluorescence was measured on a Cytek Aurora cytometer, after two washes with flow cytometry buffer. The data were analyzed with GraphPad Prism 8.0 software to determine EC50 values.

2.3 Cell-based binding assays for human PBMC-derived activated Tregs Binding specificity of the anti-CCR8 antibodies was tested by FACS using human PBMC-derived activated Tregs. In summary, Tregs were expanded and activated following the manufacturer's protocol (130-095-345, Miltenyi). Briefly, cryopreserved 7-day expanded human PBMC-derived Tregs (SailyBio) were thawed and rested overnight. They were then restimulated with CD3/CD28 MACSiBeads in the presence of rhIL-2 for 24 hours. CCR8 expression on these activated Tregs was confirmed using anti-human CCR8 (clone 433H, BD) or anti-human CCR8 (clone L263G8, Biolegend). Afterwards, the activated Tregs were plated at 20K/well in 96-well plates, spun down and the supernatant was removed. Cell pellets were then resuspended and incubated with 100 μl of various concentrations of mAbs or a human lgG1 isotype control antibody (biolegend #403501) at 37° C. for 30 min. The final concentrations of antibodies were indicated on x-axis in each figure. The cells were washed three times with FACS buffer (PBS containing 0.5 mg/ml BSA and 1 mM EDTA). PE-labeled goat-anti-human IgG (Invitrogen) diluted in FACS buffer was added to cell pellets as secondary antibodies at a concentration of 3 μg/ml, and the samples were incubated at 4° C. for 30 min. Samples were washed three times with FACS buffer and analyzed using a Cytek Aurora cytometer. Data were analyzed with GraphPad Prism 8.0 software to determine EC50 values.

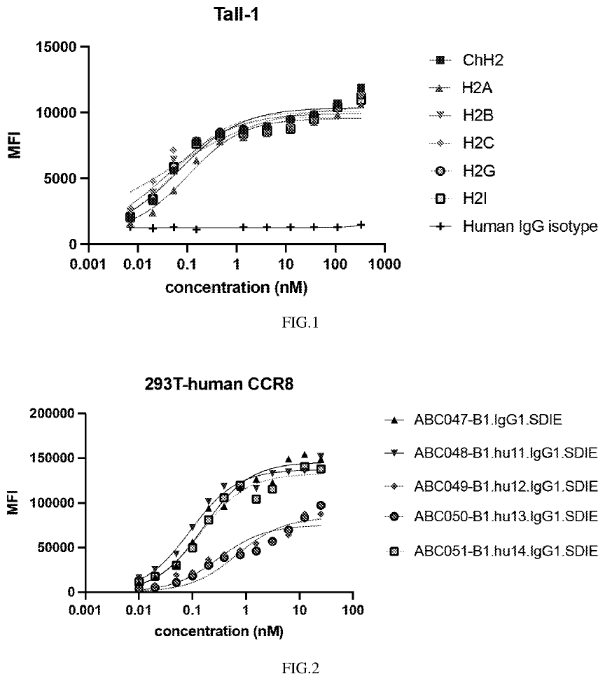

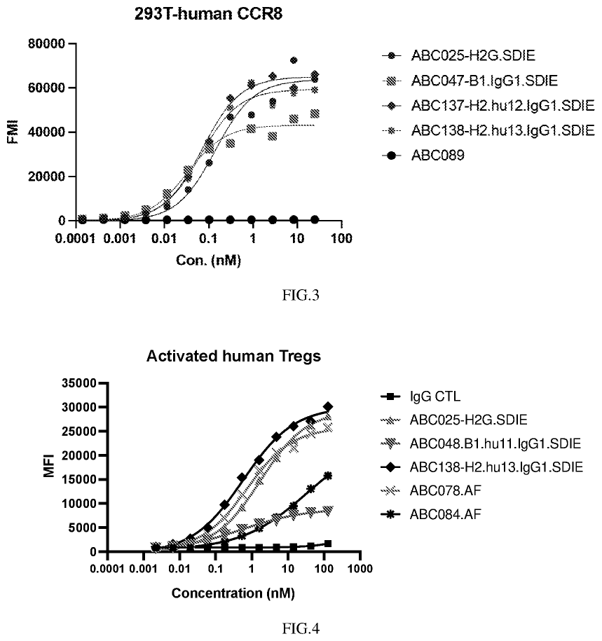

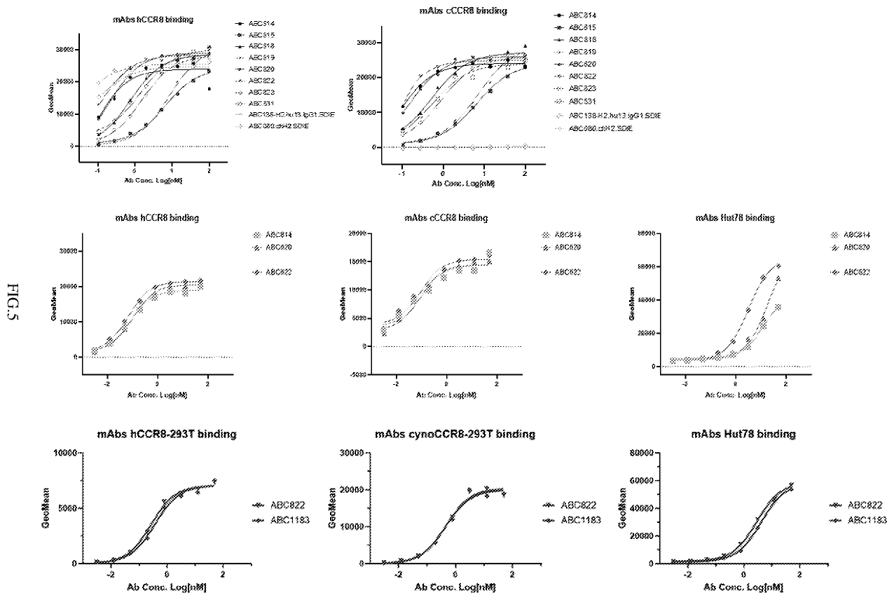

FIGS. 1-4 and Table 3 show that all the antibodies listed in Table 1 can bind to human CCR8 on the cell surface. In particular, ABC137-H2.hu12.IgG1.SDIE and ABC138-H2.hu13.IgG1.SDIE display higher affinity to CCR8-expressing cells than their parental clone ABC025.H2G.SDIE. Additionally, ABC138-H2.hu13.IgG1.SDIE exhibits the highest binding capability to the activated Tregs (FIG. 4). The negative control antibody, ABC089, with the heavy chain amino acid sequence as set forth in SEQ ID NO:178 and the light chain sequence as set forth in SEQ ID NO:179, was derived from PDB 2EIZ.

Table 3 shows affinities of anti-CCR8 mAbs binding to human PBMC-derived activated Tregs, demonstrating that the antibodies of the present disclosure are much more effective than the prior art ABC084. AF (disclosed in PCT/EP2021/067504) with the amino acid sequence of heavy chain as set forth in SEQ ID NO:52 and light chain as set forth in SEQ ID NO:53. Especially, ABC048-B1.hu11.IgG1.SDIE and ABC138-H2.hu13.IgG1.SDIE are even more effective than the prior art ABC078.AF (also known as BMS-986340 of BMS, disclosed in PCT/US2021/023430) with the amino acid sequence of heavy chain as set forth in SEQ ID NO:54 and light chain as set forth in SEQ ID NO:55, which has entered Phase I/II trial.

TABLE 3
EC50s of the anti-CCR8 antibodies binding
to human PBMC-derived activated Tregs

| mAb | EC50s (nM) |
|---|---|
| ABC025.H2G.SDIE | 1.776 |
| ABC048.B1.hu11.IgG1.SDIE | 0.5214 |
| ABC0138.H2.Hu13.SDIE | 0.5790 |
| ABC078.AF | 0.7286 |
| ABC084.AF | 33.77 |

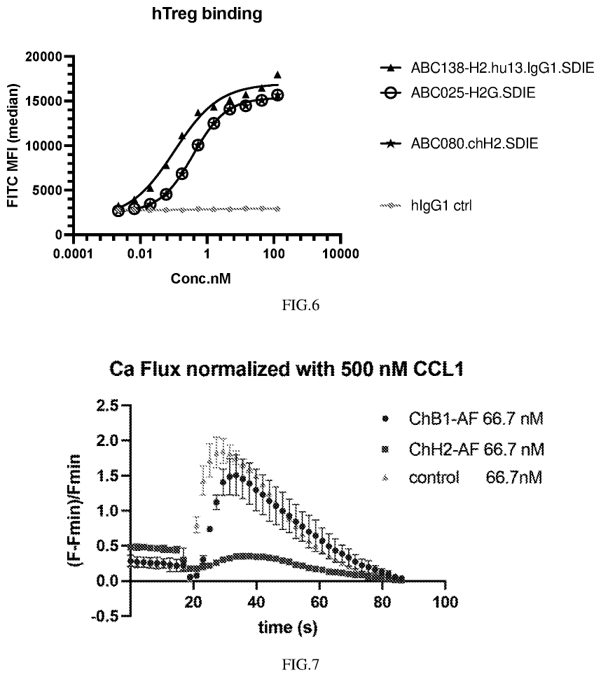

Moreover, FIGS. 5-6 and Tables 4-6 show that all the antibodies in Table 2 have the ability of binding to human CCR8 on the cell surface. Among them, ABC822 and its humanized format ABC1183 exhibit high cross-reactivity to human and cynomolgus macaque CCR8, as well as the endogenous CCR8 on Hut78 cell lines. The prior art antibody ABC026 (disclosed in PCT/US2021/017268) has the amino acid sequence of heavy chain as set forth in SEQ ID NO: 174 and light chain as set forth in SEQ ID NO:175. The antibody ABC080.chH2.SDIE has the amino acid sequence of heavy chain as set forth in SEQ ID NO:176 and light chain as set forth in SEQ ID NO:177.

TABLE 4
EC50s of antibodies binding to CCR8 on the cell lines

| | | 293T- | 293T- | 293T- |
|---|---|---|---|---|
| EC50 (nM) | Hut78 | hCCR8 | cCCR8 | mCCR8 |
| ABC814 | 11.63 | 0.08276 | 0.1005 | no binding |
| ABC815 | NA | 6.473 | 6.473 | no binding |
| ABC816 | no binding | no binding | no binding | 0.1333 |
| ABC818 | NA | 0.7436 | 0.5368 | no binding |
| ABC819 | NA | 8.507 | 7.105 | no binding |
| ABC820 | 19.45 | 0.103 | 0.09001 | no binding |
| ABC821 | no binding | no binding | no binding | 6.736 |
| ABC822 | 3.162 | 0.06837 | 0.09382 | no binding |
| ABC823 | NA | 1.511 | 0.8837 | no binding |
| ABC519 | no binding | no binding | 0.25 | no binding |
| ABC523 | no binding | no binding | 0.26 | no binding |
| ABC527 | NA | 1.74 | 2.14 | no binding |
| ABC531 | NA | 1.438 | 0.5545 | no binding |
| ABC138- | 0.1015 | 0.0237 | no binding | no binding |
| H2.hu13.IgG1.SDIE | | | | |
| ABC080.chH2.SDIE | NA | 0.135 | no binding | no binding |
| ABC026 | 0.184 | 6.508 | no binding | no binding |
| ABC078 | 0.6479 | 0.02815 | no binding | no binding |

“NA” means no data

TABLE 5
EC50s of antibodies binding to the
cell lines with CCR8 expression.

| EC50 (nM) | Hut78 | 293T-hCCR8 | 293T-cCCR8 | 293T-mCCR8 |
|---|---|---|---|---|
| ABC822 | 2.917 | 0.2689 | 0.4637 | no binding |
| ABC1183 | 4.121 | 0.3727 | 0.4448 | no binding |

Especially, FIG. 6 and Table 6 show the titration of anti-CCR8 mAbs ABC138-H2.hu13.IgG1.SDIE, ABC025-H2G.SDIE and ABC080.chH2.SDIE to human PBMC-derived activated Treg cells. ABC138-H2.hu13.IgG1.SDIE was developed by mutagenesis in the light chain CDRs of ABC025.H2G.SDIE, which is the humanized clone of ABC080.chH2.SDIE. Then the binding of anti-CCR8 monoclonal antibodies was tested using human PBMC-derived activated Tregs, as described in the FIG. 6 and Table 6, the results show that ABC138-H2.hu13.IgG1.SDIE exhibits a higher affinity than both the chimeric ABC080.chH2.SDIE and the humanized ABC025.H2G.SDIE.

TABLE 6
EC50s of anti-CCR8 mAbs binding to human
PBMC-derived activated Treg cells

| Anti-CCR8 mAb | EC50 (nM) |
|---|---|
| ABC138-H2.hu13.IgG1.SDIE | 0.1051 |
| ABC025.H2G.SDIE | 0.3799 |
| ABC080.chH2.SDIE | 0.3799 |

### Example 3: Competition Assay of the Anti-CCR8 Antibodies of the Present Disclosure with CCL1

The CC chemokine (CCL1) is a potent chemoattractant for leukocytes via its interaction with CCR8. To test the effectiveness of anti-human CCR8 antibodies in blocking this interaction, a calcium flux assay was conducted on the CHEM1-CCR8 cell line (Eurofins #HTS013RTA), which expressed high levels of human CCR8 on the cell surface, and contained the promiscuous G protein Ga15 necessary for coupling the receptor to the calcium signaling pathway. CHEM1-CCR8 cells were cultured overnight in 96-well plates at a density of 70K/well and labeled with Calbryte 520 AM (Biomol #ABD-20650). After discarding labeling buffer, 60 μl of anti-CCR8 mAbs diluted in HBSS buffer were added to the cells and incubated for 15 minutes at room temperature. Human CCL1 protein (20 μl) at 2,000 nM (Biolegend #582702) was then injected to each well to reach a final concentration of 500 nM through a Flexstation-flex3 system, and the calcium flux signal was monitored for 90 seconds using specific settings: fluorescence bottom read mode, Excitation=490 nm, Emission=525 nm, Cutoff=515 nm, time=90 s, interval=2.1 s, reads=43.

FIGS. 7-8 show that anti-human CCR8 ChH2-AF blocks the signaling of human CCL1 to human CCR8, whereas the anti-human CCR8 ChB1-AF has limited impact on the CCL1-CCR8 signaling. Studies have shown that CCLI binding to CCR8 can further enhance the suppressive activity of Tregs in in vitro assays and suppress autoimmune inflammatory responses in mouse models. Therefore, anti-CCR8 antibodies that inhibit binding of CCLI to CCR8 may have better anti-tumor efficacy, and disrupting the CCL1-CCR8 interaction may further increase anti-tumor potency of anti-CCR8 mAbs with enhanced effector function.

### Example 4: ADCC Potential of Anti-CCR8 Antibodies

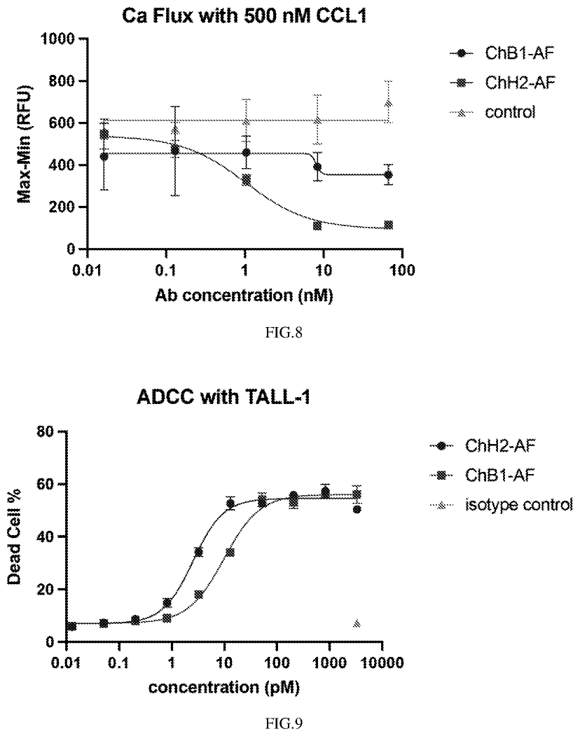

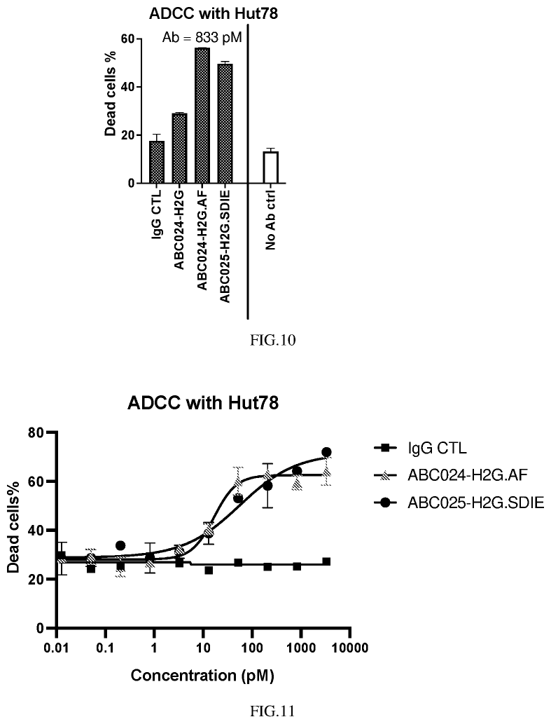

To evaluate the ADCC capability of anti-CCR8 mAbs, primary human NK cells were used as effector cells, while TALL-1 (DSMZ #ACC 521) or Hut78 (ATCC #TIB-161) were used as target cells (FIGS. 9-11). NK cells were enriched from PBMCs using a negative selection kit (Biolegend #480053) and were confirmed to be CD3-CD56+ with purities of 74% in FIG. 9, 88.8% in FIG. 10 and 81% in FIG. 11. The purified NK cells had either FcγRIIIA 158V/F or V/V genotypes.

The target cells were labeled with CellTracer violet (Thermo #C34557) and various antibodies were added to the cell mixture. In FIG. 9, 133K/well of NK and 16,625/well of TALL-1 target cells at an 8:1 ratio were used, while in FIG. 10, 160K/well of NK cells and 20K/well of Hut78 target cells at a ratio of 8:1 were used. In FIG. 11, 128K/well of NK cells and 20K/well of Hut78 target cells at a ratio of 6.4:1 were used. The NK and target cells were co-cultured with different antibodies at 37° C. for 4 hours in the U-bottom 96-well plates, and cell killing was assessed using Live/Dead Fixable Near IR dye at 1:1000 dilution (Thermo #L34994) and quantified by a Cytek Aurora cytometer with the Cytek default settings. The EC50 was calculated with the Sigmoidal 4 parameter model with PRISM.

FIG. 9 and Table 7 showed that primary NK cells can effectively mediate ADCC of CCR8 expressing cells when treated with anti-CCR8 mAbs ChH2-AF or ChB1-AF.

TABLE 7
ADCC killing of TALL-1 cells mediated
by afucosylated anti-CCR8 mAbs

| Anti-CCR8 mAb | Median EC50 (pM) |
|---|---|
| ChH2-AF | 2.546 |
| ChB1-AF | 9.799 |

The ADCC potential of anti-CCR8 mAbs, such as AB024-H2G.AF, ABC025.H2G.SDIE and ABC024-H2G, is demonstrated in FIG. 10. Fc engineering, through either afucosylation, such as AB024-H2G.AF, or SDIE mutation, such as ABC025.H2G.SDIE, results in enhanced killing of target cells in the ADCC assay when compared to ABC024-H2G with wild-type Fc.

The dose-dependent killing efficacy of ABC024-H2G.AF and ABC025-H2G.SDIE in the ADCC assay is compared in FIG. 11. The two molecules, which share the variable domains and bear different effector function enhancement technologies, demonstrate a largely comparable ADCC potential in the assay.

### 4.2 ADCC Assay Against Jurkat-h/Cyno-CCR8 or Hut78 Cells

To generate Jurkat cell lines overexpressing human or cyno CCR8, the target gene was cloned into a lentiviral transfer vector (VPK-206, Cell Biolabs). Lentiviral particles were produced in 293T cells by transient transfection of the lentiviral transfer vector carrying CCR8 and the ViraSafe Lentiviral Packaging System (Cell Biolabs #VPK-206). Jurkat cells were transduced with viral particles in the presence of 10 μg/ml polybrene, followed by treatment with 1 μg/ml of puromycin for 14 days. Overexpression of CCR8 on Jurkat cells was confirmed by flow cytometry.

For ADCC assays, PBMC effectors from healthy donors were thawed and rested overnight in ADCC medium (RPMI 1640 with glutamine, 10% low IgG heat-inactivated FBS, 1% Glutamix, 1% non-essential amino acids, 1% sodium pyruvate). The cells were then collected and washed with ADCC washing buffer (1% BSA in DPBS) to be used as effector cells.

Jurkat-hCCR8, Jurkat-cyno-CCR8 and Hut78 cells were labeled with 10,000-fold diluted CellTrace Violet Dye (Invitrogen, Cat. No. C34557) for 20 min, followed by two washes with ADCC washing buffer. The labeled cells were resuspended in ADCC medium and plated in 96-well U-bottom plates at 30,000 cells/well 50 μl/well as target cells.

After incubating the target cells with 50 μl of serially diluted antibodies for 20 minutes at room temperature, effector cells were added to the plates at an effector cell: target cell (E:T) ratio of 8. After 18 hours of treatment, the cells were stained with Live/Dead Fixable Near IR (1:1000, Invitrogen, Cat. No. L34976) for 20 minutes on ice. Cell killing was analyzed using a Cytek Aurora cytometer and the data were analyzed with GraphPad Prism 9.0 software to determine EC50.

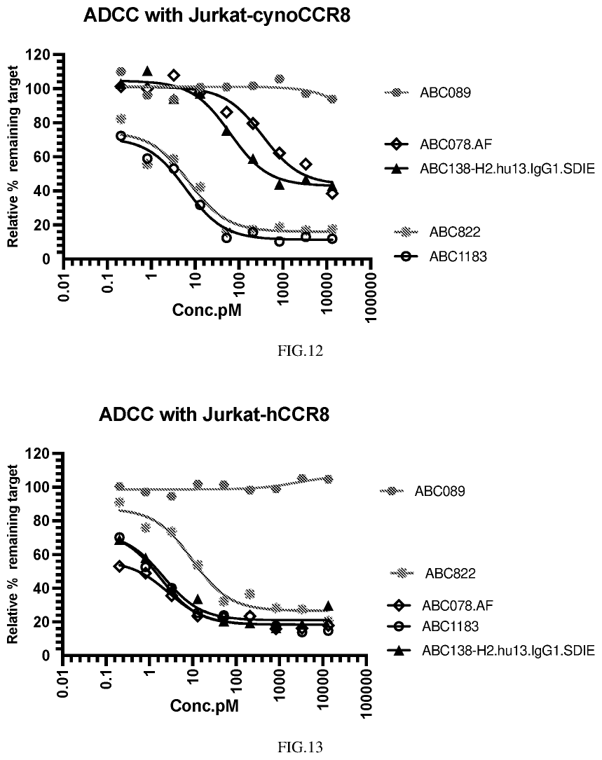

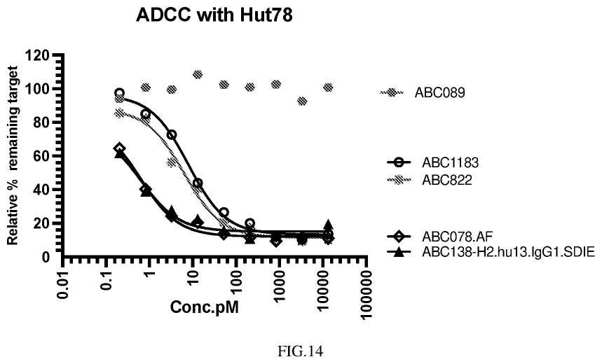

ABC138 (ABC138-H2.hu13.IgG1.SDIE) and ABC078.AF exhibit limited ADCC activity against Jurkat-cyno-CCR8, which is consistent with their minimal cross-reactivity to cyno CCR8 (FIGS. 12-14 and Tables 8-10). In contrast, ABC822 and its humanized mAb ABC1183 show potent activity in killing both Jurkat-cyno-CCR8 and Jurkat-hCCR8 cells. The negative control antibody, ABC089, with the heavy chain amino acid sequence as set forth in SEQ ID NO:178 and the light chain sequence as set forth in SEQ ID NO:179, was derived from PDB 2EIZ. Nevertheless, ABC138-H2.hu13.IgG1.SDIE demonstrates the highest activity in killing Hut78 cells with endogenous CCR8 expression.

TABLE 8
ADCC killing of Jurkat-cynoCCR8
cells mediated by anti-CCR8 mAbs

| Anti-CCR8 mAb | IC50 (pM) |
|---|---|
| ABC138-H2.hu13.IgG1.SDIE | 62.90 |
| ABC078.AF | 345.8 |
| ABC1183 | 6.150 |
| ABC822 | 7.190 |

TABLE 9
ADCC killing of Jurkat-hCCR8 cells
mediated by anti-CCR8 mAbs

| Anti-CCR8 mAb | IC50 (pM) |
|---|---|
| ABC138-H2.hu13.IgG1.SDIE | 2.088 |
| ABC078.AF | 2.788 |
| ABC1183 | 1.796 |
| ABC822 | 9.677 |

TABLE 10
ADCC killing of Hut78 cells mediated by anti-CCR8 mAbs

| Anti-CCR8 mAb | IC50 (pM) |
|---|---|
| ABC138-H2.hu13.IgG1.SDIE | 0.6519 |
| ABC078.AF | 0.6367 |
| ABC1183 | 7.945 |
| ABC822 | 6.306 |

1. An anti-CCR8 antibody or antigen-binding portion thereof comprising a heavy chain CDR1 having any one of the amino acid sequences set forth in SEQ ID NOs: 1-2, 56-60, and 62, a heavy chain CDR2 having the amino acid sequence set forth in SEQ ID NO: 3, 63-67, and 69-73, a heavy chain CDR3 having any one of the amino acid sequences set forth in SEQ ID NOs: 4-5, 74-78, and 80-85, a light chain CDR1 having any one of the amino acid sequences set forth in SEQ ID NOs: 6-11, 86-90, and 92-93, a light chain CDR2 having the amino acid sequence set forth in SEQ ID NO: 12, 94-98, and 100-102, and a light chain CDR3 having any one of the amino acid sequences set forth in SEQ ID NOs: 13-14, 103-107, and 109-113.

2. The anti-CCR8 antibody or antigen-binding portion thereof according to claim 1, wherein:
1) the heavy chain CDR3 has any one of the amino acid sequence set forth in SEQ ID NO: 4, 74-78, and 80-85; and
the light chain CDR1 has any one of the amino acid sequences set forth in SEQ ID NOs: 7, 10-11, 86-90, and 92-93;
2) the light chain CDR I has any one of the amino acid sequences set forth in SEQ ID NOs: 10-11, 86-90, and 92-93; and the light chain CDR3 has any one of the amino acid sequence set forth in SEQ ID NOs: 14, 103-107, and 109-113; or
3) the light chain CDR1 has any one of the amino acid sequence set forth in SEQ ID NOS: 10, 86-90, and 92-93.

3.-4. (canceled)

5. The anti-CCR8 antibody or antigen-binding portion thereof according to claim 1, wherein:
1) the heavy chain CDR1 has the amino acid sequence set forth in SEQ ID NO: 1, the heavy chain CDR2 has the amino acid sequence set forth in SEQ ID NO: 3, the heavy chain CDR3 has the amino acid sequence set forth in SEQ ID NO: 4, the light chain CDR1 has the amino acid sequence set forth in SEQ ID NO: 6, the light chain CDR2 has the amino acid sequence set forth in SEQ ID NO: 12, and the light chain CDR3 has the amino acid sequence set forth in SEQ ID NO: 13;
2) the heavy chain CDR1 has the amino acid sequence set forth in SEQ ID NO: 2, the heavy chain CDR2 has the amino acid sequence set forth in SEQ ID NO: 3, the heavy chain CDR3 has the amino acid sequence set forth in SEQ ID NO: 5, the light chain CDR1 has the amino acid sequence set forth in SEQ ID NO: 11, the light chain CDR2 has the amino acid sequence set forth in SEQ ID NO: 12, and the light chain CDR3 has the amino acid sequence set forth in SEQ ID NO: 14;
3) the heavy chain CDR1 has the amino acid sequence set forth in SEQ ID NO: 1, the heavy chain CDR2 has the amino acid sequence set forth in SEQ ID NO: 3, the heavy chain CDR3 has the amino acid sequence set forth in SEQ ID NO: 4, the light chain CDR1 has the amino acid sequence set forth in SEQ ID NO: 7, the light chain CDR2 has the amino acid sequence set forth in SEQ ID NO: 12, and the light chain CDR3 has the amino acid sequence set forth in SEQ ID NO: 13;
4) the heavy chain CDR1 has the amino acid sequence set forth in SEQ ID NO: 2, the heavy chain CDR2 has the amino acid sequence set forth in SEQ ID NO: 3, the heavy chain CDR3 has the amino acid sequence set forth in SEQ ID NO: 5, the light chain CDR1 has the amino acid sequence set forth in SEQ ID NO: 8, the light chain CDR2 has the amino acid sequence set forth in SEQ ID NO: 12, and the light chain CDR3 has the amino acid sequence set forth in SEQ ID NO: 14;
5) the heavy chain CDR1 has the amino acid sequence set forth in SEQ ID NO: 2, the heavy chain CDR2 has the amino acid sequence set forth in SEQ ID NO: 3, the heavy chain CDR3 has the amino acid sequence set forth in SEQ ID NO: 5, the light chain CDR1 has the amino acid sequence set forth in SEQ ID NO: 9, the light chain CDR2 has the amino acid sequence set forth in SEQ ID NO: 12, and the light chain CDR3 has the amino acid sequence set forth in SEQ ID NO: 14;
6) the heavy chain CDR1 has the amino acid sequence set forth in SEQ ID NO: 2, the heavy chain CDR2 has the amino acid sequence set forth in SEQ ID NO: 3, the heavy chain CDR3 has the amino acid sequence set forth in SEQ ID NO: 5, the light chain CDR1 has the amino acid sequence set forth in SEQ ID NO: 10, the light chain CDR2 has the amino acid sequence set forth in SEQ ID NO: 12, and the light chain CDR3 has the amino acid sequence set forth in SEQ ID NO: 14;
7) the heavy chain CDR1 has the amino acid sequence set forth in SEQ ID NO: 1, the heavy chain CDR2 has the amino acid sequence set forth in SEQ ID NO: 3, the heavy chain CDR3 has the amino acid sequence set forth in SEQ ID NO: 4, the light chain CDR1 has the amino acid sequence set forth in SEQ ID NO: 11, the light chain CDR2 has the amino acid sequence set forth in SEQ ID NO: 12, and the light chain CDR3 has the amino acid sequence set forth in SEQ ID NO: 14;
8) the heavy chain CDR1 has the amino acid sequence set forth in SEQ ID NO: 1, the heavy chain CDR2 has the amino acid sequence set forth in SEQ ID NO: 3, the heavy chain CDR3 has the amino acid sequence set forth in SEQ ID NO: 4, the light chain CDR1 has the amino acid sequence set forth in SEQ ID NO: 10, the light chain CDR2 has the amino acid sequence set forth in SEQ ID NO: 12, and the light chain CDR3 has the amino acid sequence set forth in SEQ ID NO: 14;
9) the heavy chain CDR1 has the amino acid sequence set forth in SEQ ID NO: 56 the heavy chain CDR2 has the amino acid sequence set forth in SEQ ID NO: 63, the heavy chain CDR3 has the amino acid sequence set forth in SEQ ID NO: 74, the light chain CDR1 has the amino acid sequence set forth in SEQ ID NO: 86, the light chain CDR2 has the amino acid sequence set forth in SEQ ID NO: 94, and the light chain CDR3 has the amino acid sequence set forth in SEQ ID NO: 103;
10) the heavy chain CDR1 has the amino acid sequence set forth in SEQ ID NO: 57, the heavy chain CDR2 has the amino acid sequence set forth in SEQ ID NO: 64, the heavy chain CDR3 has the amino acid sequence set forth in SEQ ID NO: 75, the light chain CDR1 has the amino acid sequence set forth in SEQ ID NO: 87, the light chain CDR2 has the amino acid sequence set forth in SEQ ID NO: 95, and the light chain CDR3 has the amino acid sequence set forth in SEQ ID NO: 104;
11) the heavy chain CDR1 has the amino acid sequence set forth in SEQ ID NO: 56, the heavy chain CDR2 has the amino acid sequence set forth in SEQ ID NO: 63, the heavy chain CDR3 has the amino acid sequence set forth in SEQ ID NO: 74, the light chain CDR1 has the amino acid sequence set forth in SEQ ID NO: 86, the light chain CDR2 has the amino acid sequence set forth in SEQ ID NO: 94, and the light chain CDR3 has the amino acid sequence set forth in SEQ ID NO: 103;
12) the heavy chain CDR1 has the amino acid sequence set forth in SEQ ID NO: 57, the heavy chain CDR2 has the amino acid sequence set forth in SEQ ID NO: 64, the heavy chain CDR3 has the amino acid sequence set forth in SEQ ID NO: 75, the light chain CDR1 has the amino acid sequence set forth in SEQ ID NO: 87, the light chain CDR2 has the amino acid sequence set forth in SEQ ID NO: 95, and the light chain CDR3 has the amino acid sequence set forth in SEQ ID NO: 104;
13) the heavy chain CDR1 has the amino acid sequence set forth in SEQ ID NO: 58, the heavy chain CDR2 has the amino acid sequence set forth in SEQ ID NO: 65, the heavy chain CDR3 has the amino acid sequence set forth in SEQ ID NO: 76, the light chain CDR1 has the amino acid sequence set forth in SEQ ID NO: 88, the light chain CDR2 has the amino acid sequence set forth in SEQ ID NO: 96, and the light chain CDR3 has the amino acid sequence set forth in SEQ ID NO: 105;
14) the heavy chain CDR1 has the amino acid sequence set forth in SEQ ID NO: 59, the heavy chain CDR2 has the amino acid sequence set forth in SEQ ID NO: 66, the heavy chain CDR3 has the amino acid sequence set forth in SEQ ID NO: 77, the light chain CDR1 has the amino acid sequence set forth in SEQ ID NO: 89, the light chain CDR2 has the amino acid sequence set forth in SEQ ID NO: 97, and the light chain CDR3 has the amino acid sequence set forth in SEQ ID NO: 106;
15) the heavy chain CDR1 has the amino acid sequence set forth in SEQ ID NO: 60, the heavy chain CDR2 has the amino acid sequence set forth in SEQ ID NO: 67, the heavy chain CDR3 has the amino acid sequence set forth in SEQ ID NO: 78, the light chain CDR1 has the amino acid sequence set forth in SEQ ID NO: 90, the light chain CDR2 has the amino acid sequence set forth in SEQ ID NO: 98, and the light chain CDR3 has the amino acid sequence set forth in SEQ ID NO: 107;
16) the heavy chain CDR1 has the amino acid sequence set forth in SEQ ID NO: 58, the heavy chain CDR2 has the amino acid sequence set forth in SEQ ID NO: 69, the heavy chain CDR3 has the amino acid sequence set forth in SEQ ID NO: 80, the light chain CDR1 has the amino acid sequence set forth in SEQ ID NO: 88, the light chain CDR2 has the amino acid sequence set forth in SEQ ID NO: 100, and the light chain CDR3 has the amino acid sequence set forth in SEQ ID NO: 109;
17) the heavy chain CDR1 has the amino acid sequence set forth in SEQ ID NO: 58, the heavy chain CDR2 has the amino acid sequence set forth in SEQ ID NO: 70, the heavy chain CDR3 has the amino acid sequence set forth in SEQ ID NO: 81, the light chain CDR1 has the amino acid sequence set forth in SEQ ID NO: 88, the light chain CDR2 has the amino acid sequence set forth in SEQ ID NO: 100, and the light chain CDR3 has the amino acid sequence set forth in SEQ ID NO: 109;
18) the heavy chain CDR1 has the amino acid sequence set forth in SEQ ID NO: 58, the heavy chain CDR2 has the amino acid sequence set forth in SEQ ID NO: 65, the heavy chain CDR3 has the amino acid sequence set forth in SEQ ID NO: 82, the light chain CDR1 has the amino acid sequence set forth in SEQ ID NO: 88, the light chain CDR2 has the amino acid sequence set forth in SEQ ID NO: 101, and the light chain CDR3 has the amino acid sequence set forth in SEQ ID NO: 110;
19) the heavy chain CDR1 has the amino acid sequence set forth in SEQ ID NO: 62, the heavy chain CDR2 has the amino acid sequence set forth in SEQ ID NO: 71, the heavy chain CDR3 has the amino acid sequence set forth in SEQ ID NO: 83, the light chain CDR1 has the amino acid sequence set forth in SEQ ID NO: 92, the light chain CDR2 has the amino acid sequence set forth in SEQ ID NO: 102, and the light chain CDR3 has the amino acid sequence set forth in SEQ ID NO: 111;
20) the heavy chain CDR1 has the amino acid sequence set forth in SEQ ID NO: 58, the heavy chain CDR2 has the amino acid sequence set forth in SEQ ID NO: 72, the heavy chain CDR3 has the amino acid sequence set forth in SEQ ID NO: 84, the light chain CDR1 has the amino acid sequence set forth in SEQ ID NO: 93, the light chain CDR2 has the amino acid sequence set forth in SEQ ID NO: 100, and the light chain CDR3 has the amino acid sequence set forth in SEQ ID NO: 112; or
21) the heavy chain CDR1 has the amino acid sequence set forth in SEQ ID NO: 58, the heavy chain CDR2 has the amino acid sequence set forth in SEQ ID NO: 73, the heavy chain CDR3 has the amino acid sequence set forth in SEQ ID NO: 85, the light chain CDR1 has the amino acid sequence set forth in SEQ ID NO: 88, the light chain CDR2 has the amino acid sequence set forth in SEQ ID NO: 100, and the light chain CDR3 has the amino acid sequence set forth in SEQ ID NO: 113.

6. The anti-CCR8 antibody or antigen-binding portion thereof according to claim 1, comprising a variable region of heavy chain and a variable region of light chain, wherein
the variable region of heavy chain has any one of the amino acid sequences set forth in SEQ ID NOs: 15-20, 114-120, and 122-128; or
the variable region of heavy chain has any one of the amino acid sequences set forth in SEQ ID NOs: 16-20, 114-120, and 122-128; or
the variable region of heavy chain has any one of the amino acid sequence set forth in SEQ ID NO: 18, 114-120, and 122-128; or
the variable region of light chain has any one of the amino acid sequences set forth in SEQ ID NOs: 21-28, 129-135, and 137-143; or
the variable region of light chain has any one of the amino acid sequences set forth in SEQ ID NOs: 22-28, 129-135, and 137-143; or
the variable region of light chain has any one of the amino acid sequences set forth in SEQ ID NOs: 24, 25, 28, 129-135, and 137-143; or
the variable region of light chain has any one of the amino acid sequences set forth in SEQ ID NOs: 25, 28, 129-135, and 137-143; or
the variable region of light chain has any one of the amino acid sequence set forth in SEQ ID NO: 28, 129-135, and 137-143.

7.-13. (canceled)

14. The anti-CCR8 antibody or antigen-binding portion thereof according to claim 1, wherein:
1) the variable region of heavy chain has the amino acid sequence set forth in SEQ ID NO: 15 and the variable region of light chain has the amino acid sequence set forth in SEQ ID NO: 21;
2) the variable region of heavy chain has the amino acid sequence set forth in SEQ ID NO: 16 and the variable region of light chain has the amino acid sequence set forth in SEQ ID NO: 22;
3) the variable region of heavy chain has the amino acid sequence set forth in SEQ ID NO: 17 and the variable region of light chain has the amino acid sequence set forth in SEQ ID NO: 23;
4) the variable region of heavy chain has the amino acid sequence set forth in SEQ ID NO: 18 and the variable region of light chain has the amino acid sequence set forth in SEQ ID NO: 23;
5) the variable region of heavy chain has the amino acid sequence set forth in SEQ ID NO: 19 and the variable region of light chain has the amino acid sequence set forth in SEQ ID NO: 23;
6) the variable region of heavy chain has the amino acid sequence set forth in SEQ ID NO: 18 and the variable region of light chain has the amino acid sequence set forth in SEQ ID NO: 24;
7) the variable region of heavy chain has the amino acid sequence set forth in SEQ ID NO: 19 and the variable region of light chain has the amino acid sequence set forth in SEQ ID NO: 24;
8) the variable region of heavy chain has the amino acid sequence set forth in SEQ ID NO: 20 and the variable region of light chain has the amino acid sequence set forth in SEQ ID NO: 25;
9) the variable region of heavy chain has the amino acid sequence set forth in SEQ ID NO: 20 and the variable region of light chain has the amino acid sequence set forth in SEQ ID NO: 26;
10) the variable region of heavy chain has the amino acid sequence set forth in SEQ ID NO: 20 and the variable region of light chain has the amino acid sequence set forth in SEQ ID NO: 27;
11) the variable region of heavy chain has the amino acid sequence set forth in SEQ ID NO: 20 and the variable region of light chain has the amino acid sequence set forth in SEQ ID NO: 28;
12) the variable region of heavy chain has the amino acid sequence set forth in SEQ ID NO: 18 and the variable region of light chain has the amino acid sequence set forth in SEQ ID NO: 25;
13) the variable region of heavy chain has the amino acid sequence set forth in SEQ ID NO: 18 and the variable region of light chain has the amino acid sequence set forth in SEQ ID NO: 28;
14) the variable region of heavy chain has the amino acid sequence set forth in SEQ ID NO: 114 and the variable region of light chain has the amino acid sequence set forth in SEQ ID NO: 129;
15) the variable region of heavy chain has the amino acid sequence set forth in SEQ ID NO: 115 and the variable region of light chain has the amino acid sequence set forth in SEQ ID NO: 130;
16) the variable region of heavy chain has the amino acid sequence set forth in SEQ ID NO: 116 and the variable region of light chain has the amino acid sequence set forth in SEQ ID NO: 131;
17) the variable region of heavy chain has the amino acid sequence set forth in SEQ ID NO: 117 and the variable region of light chain has the amino acid sequence set forth in SEQ ID NO: 132;
18) the variable region of heavy chain has the amino acid sequence set forth in SEQ ID NO: 118 and the variable region of light chain has the amino acid sequence set forth in SEQ ID NO: 133;
19) the variable region of heavy chain has the amino acid sequence set forth in SEQ ID NO: 119 and the variable region of light chain has the amino acid sequence set forth in SEQ ID NO: 134;
20) the variable region of heavy chain has the amino acid sequence set forth in SEQ ID NO: 120 and the variable region of light chain has the amino acid sequence set forth in SEQ ID NO: 135;
21) the variable region of heavy chain has the amino acid sequence set forth in SEQ ID NO: 122 and the variable region of light chain has the amino acid sequence set forth in SEQ ID NO: 137;
22) the variable region of heavy chain has the amino acid sequence set forth in SEQ ID NO: 123 and the variable region of light chain has the amino acid sequence set forth in SEQ ID NO: 138;
23) the variable region of heavy chain has the amino acid sequence set forth in SEQ ID NO: 124 and the variable region of light chain has the amino acid sequence set forth in SEQ ID NO: 139;
24) the variable region of heavy chain has the amino acid sequence set forth in SEQ ID NO: 125 and the variable region of light chain has the amino acid sequence set forth in SEQ ID NO: 140;
25) the variable region of heavy chain has the amino acid sequence set forth in SEQ ID NO: 126 and the variable region of light chain has the amino acid sequence set forth in SEQ ID NO: 141;
26) the variable region of heavy chain has the amino acid sequence set forth in SEQ ID NO: 127 and the variable region of light chain has the amino acid sequence set forth in SEQ ID NO: 142; or
27) the variable region of heavy chain has the amino acid sequence set forth in SEQ ID NO: 127 and the variable region of light chain has the amino acid sequence set forth in SEQ ID NO: 143.

15. The anti-CCR8 antibody or antigen-binding portion thereof according to claim 1, comprising a constant region of heavy chain and a constant region of light chain, wherein
the constant region of heavy chain has any one of the amino acid sequences set forth in SEQ ID NOs: 29-30, optionally, the constant region of heavy chain has the amino acid sequence set forth in SEQ ID NO: 30, and/or
the constant region of light chain has the amino acid sequence set forth in SEQ ID NO: 31.

16.-18. (canceled)

19. The anti-CCR8 antibody or antigen-binding portion thereof according to claim 1, comprising a heavy chain, wherein the heavy chain has any one of the amino acid sequences set forth in SEQ ID NOs: 32-39, 144-150, and 152-158;
the heavy chain has any one of the amino acid sequences set forth in SEQ ID NOs: 34-39, 144-150, and 152-158; or
the heavy chain has any one of the amino acid sequence set forth in SEQ ID NO: 37, 144-150, and 152-158.

20.-21. (canceled)

22. The anti-CCR8 antibody or antigen-binding portion thereof according to claim 1, comprising a light chain, wherein the light chain has any one of the amino acid sequences set forth in SEQ ID NOs: 40-47, 159-165, and 167-173;
the light chain has any one of the amino acid sequences set forth in SEQ ID NOs: 41-47, 159-165, and 167-173;
the light chain has any one of the amino acid sequences set forth in SEQ ID NOs: 43, 44 and 47, 159-165, and 167-173;
the light chain has any one of the amino acid sequences set forth in SEQ ID NOs: 44 and 47, 159-165, and 167-173; or
the light chain has any one of the amino acid sequence set forth in SEQ ID NO: 47, 159-165, and 167-173.

23.-26. (canceled)

27. The anti-CCR8 antibody or antigen-binding portion thereof according to claim 1, wherein:
1) the heavy chain has the amino acid sequence set forth in SEQ ID NO: 32 and the light chain has the amino acid sequence set forth in SEQ ID NO: 40;
2) the heavy chain has the amino acid sequence set forth in SEQ ID NO: 33 and the light chain has the amino acid sequence set forth in SEQ ID NO: 41;
3) the heavy chain has the amino acid sequence set forth in SEQ ID NO: 34 and the light chain has the amino acid sequence set forth in SEQ ID NO: 42;
4) the heavy chain has the amino acid sequence set forth in SEQ ID NO: 35 and the light chain has the amino acid sequence set forth in SEQ ID NO: 42;
5) the heavy chain has the amino acid sequence set forth in SEQ ID NO: 36 and the light chain has the amino acid sequence set forth in SEQ ID NO: 42;
6) the heavy chain has the amino acid sequence set forth in SEQ ID NO: 35 and the light chain has the amino acid sequence set forth in SEQ ID NO: 43;
7) the heavy chain has the amino acid sequence set forth in SEQ ID NO: 36 and the light chain has the amino acid sequence set forth in SEQ ID NO: 43;
8) the heavy chain has the amino acid sequence set forth in SEQ ID NO: 38 and the light chain has the amino acid sequence set forth in SEQ ID NO: 41;
9) the heavy chain has the amino acid sequence set forth in SEQ ID NO: 39 and the light chain has the amino acid sequence set forth in SEQ ID NO: 44;
10) the heavy chain has the amino acid sequence set forth in SEQ ID NO: 39 and the light chain has the amino acid sequence set forth in SEQ ID NO: 45;
11) the heavy chain has the amino acid sequence set forth in SEQ ID NO: 39 and the light chain has the amino acid sequence set forth in SEQ ID NO: 46;
12) the heavy chain has the amino acid sequence set forth in SEQ ID NO: 39 and the light chain has the amino acid sequence set forth in SEQ ID NO: 47;
13) the heavy chain has the amino acid sequence set forth in SEQ ID NO: 37 and the light chain has the amino acid sequence set forth in SEQ ID NO: 43;
14) the heavy chain has the amino acid sequence set forth in SEQ ID NO: 37 and the light chain has the amino acid sequence set forth in SEQ ID NO: 44;
15) the heavy chain has the amino acid sequence set forth in SEQ ID NO: 37 and the light chain has the amino acid sequence set forth in SEQ ID NO: 47;
16) the heavy chain has the amino acid sequence set forth in SEQ ID NO: 144 and the light chain has the amino acid sequence set forth in SEQ ID NO: 159;
17) the heavy chain has the amino acid sequence set forth in SEQ ID NO: 145 and the light chain has the amino acid sequence set forth in SEQ ID NO: 160;
18) the heavy chain has the amino acid sequence set forth in SEQ ID NO: 146 and the light chain has the amino acid sequence set forth in SEQ ID NO: 161;
19) the heavy chain has the amino acid sequence set forth in SEQ ID NO: 147 and the light chain has the amino acid sequence set forth in SEQ ID NO: 162;
20) the heavy chain has the amino acid sequence set forth in SEQ ID NO: 148 and the light chain has the amino acid sequence set forth in SEQ ID NO: 163;
21) the heavy chain has the amino acid sequence set forth in SEQ ID NO: 149 and the light chain has the amino acid sequence set forth in SEQ ID NO: 164;
22) the heavy chain has the amino acid sequence set forth in SEQ ID NO: 150 and the light chain has the amino acid sequence set forth in SEQ ID NO: 165;
23) the heavy chain has the amino acid sequence set forth in SEQ ID NO: 152 and the light chain has the amino acid sequence set forth in SEQ ID NO: 167;
24) the heavy chain has the amino acid sequence set forth in SEQ ID NO: 153 and the light chain has the amino acid sequence set forth in SEQ ID NO: 168;
25) the heavy chain has the amino acid sequence set forth in SEQ ID NO: 154 and the light chain has the amino acid sequence set forth in SEQ ID NO: 169;
26) the heavy chain has the amino acid sequence set forth in SEQ ID NO: 155 and the light chain has the amino acid sequence set forth in SEQ ID NO: 170;
27) the heavy chain has the amino acid sequence set forth in SEQ ID NO: 156 and the light chain has the amino acid sequence set forth in SEQ ID NO: 171;
28) the heavy chain has the amino acid sequence set forth in SEQ ID NO: 157 and the light chain has the amino acid sequence set forth in SEQ ID NO: 172; or
29) the heavy chain has the amino acid sequence set forth in SEQ ID NO: 158 and the light chain has the amino acid sequence set forth in SEQ ID NO: 173.

28. The anti-CCR8 antibody or antigen-binding portion thereof according to claim 1, which is afucosylated;
which induces antibody-dependent cellular cytotoxicity (ADCC) in a subject following administration;
which is capable of inducing activation of NK cells;
which is capable of inducing NK cell mediated killing of tumor infiltrating Treg cells in the subject; or
which induces internalization of CCR8 by tumor infiltrating Treg cells.

29.-32. (canceled)

33. The anti-CCR8 antibody or antigen-binding portion thereof according to claim 1, which is a human antibody, a humanized antibody, or a chimeric antibody;
which is an IgG1, IgG2, IgG3, or IgG4 isotype antibody or antigen binding fragment thereof; or
which is a monoclonal antibody, a bispecific antibody, a bispecific T cell engager (BiTE), a multispecific antibody, a biparatopic antibody, an immunoconjugate, an antibody drug conjugate, a chimeric antigen receptor (CAR), a T cell receptor (TCR) or any combination thereof.

34.-35. (canceled)

36. The anti-CCR8 antibody or antigen-binding portion thereof according to claim 1, which targets the extracellular domain of human CCR8;
which specifically binds to one or more amino acids within the N-terminal extracellular domain of CCR8; or
which competes for binding with CCL1.

37. The anti-CCR8 antibody or antigen-binding portion thereof according to claim 1, which binds cells expressing CCR8 on their surface; optionally the cells are regulatory T cells (Tregs) or tumor infiltrating Tregs.

38.-41. (canceled)

42. A pharmaceutical composition comprising the anti-CCR8 antibody or antigen-binding portion thereof according to claim 1 and a pharmaceutically acceptable carrier.

43. A method of treating a disease or condition in a subject in need thereof, comprising administering to the subject the anti-CCR8 antibody or antigen-binding portion according to claim 1.

44. The method according to claim 43, wherein the disease or condition comprises tumor;
optionally, the tumor comprises Kaposi's sarcoma, leukemia, acute lymphocytic leukemia, acute myelocytic leukemia, myeloblasts promyelocyte myelomonocytic monocytic erythroleukemia chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas fibrosarcoma myxosarcoma, liposarcoma, chondrosarcoma, osteogenic sarcoma, osteosarcoma, chordoma, angiosarcoma endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma (HCC) hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma oligodendroglioma, menangioma melanoma, neuroblastoma retinoblastoma, nasopharyngeal carcinoma, esophageal carcinoma, basal cell carcinoma, biliary tract cancer bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx) ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system, and any combination thereof.

45. (canceled)

46. The method according to claim 43, wherein the tumor is refractory or relapsed, or the tumor is advanced, locally advanced, or metastatic.

47. (canceled)

48. The method according to claim 43, which further comprises administering an additional anticancer agent.

49.-53. (canceled)


SEQUENCE LISTING (179 sequences)

SEQ ID NO: 1 - protein, 5 aa, synthetic construct
TFAMN

SEQ ID NO: 2 - protein, 5 aa, synthetic construct
TYAMN

SEQ ID NO: 3 - protein, 19 aa, synthetic construct
RIRSKSNNYATYYADSVKD

SEQ ID NO: 4 - protein, 12 aa, synthetic construct
GKENNYGYAMDY

SEQ ID NO: 5 - protein, 10 aa, synthetic construct
EVRRDYALDQ

SEQ ID NO: 6 - protein, 16 aa, synthetic construct
RSSQSLLHSNGNTYLY

SEQ ID NO: 7 - protein, 16 aa, synthetic construct
RSSQSLLHSNANTYLY

SEQ ID NO: 8 - protein, 16 aa, synthetic construct
RSSKSLLHSQGNTYLY

SEQ ID NO: 9 - protein, 16 aa, synthetic construct
RSSKSLLHSSGNTYLY

SEQ ID NO: 10 - protein, 16 aa, synthetic construct
RSSKSLLHSNANTYLY

SEQ ID NO: 11 - protein, 16 aa, synthetic construct
RSSKSLLHSNGNTYLY

SEQ ID NO: 12 - protein, 7 aa, synthetic construct
RMSNLAS

SEQ ID NO: 13 - protein, 9 aa, synthetic construct
MQHLEYPFT

SEQ ID NO: 14 - protein, 9 aa, synthetic construct
MQHVEYPFT

SEQ ID NO: 15 - protein, 123 aa, synthetic construct
EVQLVETGGGLVQPKGSLRLSCAASGFPFNTFAMNWVRQAPGKGLEWLARIRSKSNNYATYYADSVKDRFTISRDDSRRMLYLLMNNVKFEDTAIYYCVRGKENNYGYAMDYWGQGTSVTVSS

SEQ ID NO: 16 - protein, 121 aa, synthetic construct
EVQLVESGGGLVQPKGSSKLSCAASGFTFNTYAMNWVRQAPGKGLEWVARIRSKSNNYATYYADSVKDRFTISRDDSQSMLYLQMNNLKTEDTAMYYCVREVRRDYALDQWGQGTSVTVSS

SEQ ID NO: 17 - protein, 123 aa, synthetic construct
EVQLVESGGGLVQPGGSLRLSCAASGFPFNTFAMNWVRQAPGKGLEWLARIRSKSNNYATYYADSVKDRFTISRDNAKNSLYLQMNSLRAEDTAVYYCVRGKENNYGYAMDYWGQGTTVTVSS

SEQ ID NO: 18 - protein, 123 aa, synthetic construct
EVQLVESGGGLVQPGGSLRLSCAASGFPFNTFAMNWVRQAPGKGLEWLARIRSKSNNYATYYADSVKDRFTISRDNAKRSLYLQMNSLRAEDTAVYYCVRGKENNYGYAMDYWGQGTTVTVSS

SEQ ID NO: 19 - protein, 123 aa, synthetic construct
EVQLVESGGGLVQPGGSLRLSCAASGFPFNTFAMNWVRQAPGKGLEWLARIRSKSNNYATYYADSVKDRFTISRDNARRSLYLQMNSLRAEDTAVYYCVRGKENNYGYAMDYWGQGTTVTVSS

SEQ ID NO: 20 - protein, 121 aa, synthetic construct
EVQLVESGGGLVQPGGSLKLSCAASGFTFNTYAMNWVRQASGKGLEWVARIRSKSNNYATYYADSVKDRFTISRDDSKNTLYLQMNSLKTEDTAVYYCVREVRRDYALDQWGQGTMVTVSS

SEQ ID NO: 21 - protein, 112 aa, synthetic construct
DIVMTQAAPSVPVTPGESVSISCRSSQSLLHSNGNTYLYWFLQRPGQSPQLLIYRMSNLASGVPDRFSGSGSGTAFTLRISRVEAEDVGVYYCMQHLEYPFTFGIGTKLELK

SEQ ID NO: 22 - protein, 112 aa, synthetic construct
DIVMTQAAPSVVVTPGESVSISCRSSKSLLHSNGNTYLYWFLQRPGQSPQLLIYRMSNLASGVPDRFSGSGSGTAFTLRISRVEAEDVGVYYCMQHVEYPFTFGGGTKVQIK

SEQ ID NO: 23 - protein, 112 aa, synthetic construct
DIVMTQSPLSLPVTPGEPASISCRSSQSLLHSNGNTYLYWFLQKPGQSPQLLIYRMSNLASGVPDRFSGSGSGTDFTLKISRVEAEDVGVYYCMQHLEYPFTFGQGTKLEIK

SEQ ID NO: 24 - protein, 112 aa, synthetic construct
DIVMTQSPLSLPVTPGEPASISCRSSQSLLHSNANTYLYWFLQKPGQSPQLLIYRMSNLASGVPDRFSGSGSGTDFTLKISRVEAEDVGVYYCMQHLEYPFTFGQGTKLEIK

SEQ ID NO: 25 - protein, 112 aa, synthetic construct
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHVEYPFTFGGGTKLEIK

SEQ ID NO: 26 - protein, 112 aa, synthetic construct
DIVMTQSPLSLPVTPGEPASISCRSSKSLLHSQGNTYLYWFLQKPGQSPQLLIYRMSNLASGVPDRFSGSGSGTDFTLKISRVEAEDVGVYYCMQHVEYPFTFGGGTKLEIK

SEQ ID NO: 27 - protein, 112 aa, synthetic construct
DIVMTQSPLSLPVTPGEPASISCRSSKSLLHSSGNTYLYWFLQKPGQSPQLLIYRMSNLASGVPDRFSGSGSGTDFTLKISRVEAEDVGVYYCMQHVEYPFTFGGGTKLEIK

SEQ ID NO: 28 - protein, 112 aa, synthetic construct
DIVMTQSPLSLPVTPGEPASISCRSSKSLLHSNANTYLYWFLQKPGQSPQLLIYRMSNLASGVPDRFSGSGSGTDFTLKISRVEAEDVGVYYCMQHVEYPFTFGGGTKLEIK

SEQ ID NO: 29 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 107 aa, synthetic construct [sequence omitted]

SEQ ID NO: 32 - protein, 453 aa, synthetic construct
EVQLVETGGGLVQPKGSLRLSCAASGFPFNTFAMNWVRQAPGKGLEWLARIRSKSNNYATYYADSVKDRFTISRDDSRRMLYLLMNNVKFEDTAIYYCVRGKENNYGYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 451 aa, synthetic construct
EVQLVESGGGLVQPKGSSKLSCAASGFTFNTYAMNWVRQAPGKGLEWVARIRSKSNNYATYYADSVKDRFTISRDDSQSMLYLQMNNLKTEDTAMYYCVREVRRDYALDQWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 453 aa, synthetic construct
EVQLVESGGGLVQPGGSLRLSCAASGFPFNTFAMNWVRQAPGKGLEWLARIRSKSNNYATYYADSVKDRFTISRDNAKNSLYLQMNSLRAEDTAVYYCVRGKENNYGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 453 aa, synthetic construct
EVQLVESGGGLVQPGGSLRLSCAASGFPFNTFAMNWVRQAPGKGLEWLARIRSKSNNYATYYADSVKDRFTISRDNAKRSLYLQMNSLRAEDTAVYYCVRGKENNYGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 453 aa, synthetic construct
EVQLVESGGGLVQPGGSLRLSCAASGFPFNTFAMNWVRQAPGKGLEWLARIRSKSNNYATYYADSVKDRFTISRDNARRSLYLQMNSLRAEDTAVYYCVRGKENNYGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 453 aa, synthetic construct
EVQLVESGGGLVQPGGSLRLSCAASGFPFNTFAMNWVRQAPGKGLEWLARIRSKSNNYATYYADSVKDRFTISRDNAKRSLYLQMNSLRAEDTAVYYCVRGKENNYGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 451 aa, synthetic construct
EVQLVESGGGLVQPKGSSKLSCAASGFTFNTYAMNWVRQAPGKGLEWVARIRSKSNNYATYYADSVKDRFTISRDDSQSMLYLQMNNLKTEDTAMYYCVREVRRDYALDQWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 451 aa, synthetic construct
EVQLVESGGGLVQPGGSLKLSCAASGFTFNTYAMNWVRQASGKGLEWVARIRSKSNNYATYYADSVKDRFTISRDDSKNTLYLQMNSLKTEDTAVYYCVREVRRDYALDQWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 219 aa, synthetic construct
DIVMTQAAPSVPVTPGESVSISCRSSQSLLHSNGNTYLYWFLQRPGQSPQLLIYRMSNLASGVPDRFSGSGSGTAFTLRISRVEAEDVGVYYCMQHLEYPFTFGIGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 219 aa, synthetic construct
DIVMTQAAPSVVVTPGESVSISCRSSKSLLHSNGNTYLYWFLQRPGQSPQLLIYRMSNLASGVPDRFSGSGSGTAFTLRISRVEAEDVGVYYCMQHVEYPFTFGGGTKVQIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 219 aa, synthetic construct
DIVMTQSPLSLPVTPGEPASISCRSSQSLLHSNGNTYLYWFLQKPGQSPQLLIYRMSNLASGVPDRFSGSGSGTDFTLKISRVEAEDVGVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 219 aa, synthetic construct
DIVMTQSPLSLPVTPGEPASISCRSSQSLLHSNANTYLYWFLQKPGQSPQLLIYRMSNLASGVPDRFSGSGSGTDFTLKISRVEAEDVGVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 219 aa, synthetic construct
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHVEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 219 aa, synthetic construct
DIVMTQSPLSLPVTPGEPASISCRSSKSLLHSQGNTYLYWFLQKPGQSPQLLIYRMSNLASGVPDRFSGSGSGTDFTLKISRVEAEDVGVYYCMQHVEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 219 aa, synthetic construct
DIVMTQSPLSLPVTPGEPASISCRSSKSLLHSSGNTYLYWFLQKPGQSPQLLIYRMSNLASGVPDRFSGSGSGTDFTLKISRVEAEDVGVYYCMQHVEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 219 aa, synthetic construct
DIVMTQSPLSLPVTPGEPASISCRSSKSLLHSNANTYLYWFLQKPGQSPQLLIYRMSNLASGVPDRFSGSGSGTDFTLKISRVEAEDVGVYYCMQHVEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 717 bp, synthetic construct
  source 1..717: mol_type = other DNA
atggtgtccaagggcgaagaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgcttcagcagataccccgaccatatgaagcagcacgacttcttcaagagcgccatgcctgagggctacgtgcaagagcggaccatcttctttaaggacgacggcaactacaagaccagggccgaagtgaagttcgagggcgacaccctggtcaaccggatcgagctgaagggcatcgacttcaaagaggacggcaacatcctgggccacaagctcgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgagcaaggaccccaacgagaagagggatcacatggtgctgctggaattcgtgaccgccgctggcatcacactcggcatggatgagctgtacaag

SEQ ID NO: 49 - DNA, 84 bp, synthetic construct
  source 1..84: mol_type = other DNA
agaagaaaacgggcccctgtgaagcagaccctgaacttcgatctgctgaagctggccggcgacgtggaaagcaatcctggacct

SEQ ID NO: 50 - DNA, 1065 bp, synthetic construct
  source 1..1065: mol_type = other DNA
atggactacaccctggacctgagcgtgaccaccgtgaccgactactactaccccgacatcttcagcagcccttgcgacgccgagctgatccagacaaatggcaaactgctgctggccgtgttctactgcctgctgttcgtgtttagcctgctgggcaacagcctggtcatcctggttctggtcgtgtgcaagaagctgcggagcatcaccgacgtgtacctgctgaatctggccctgagcgacctgctctttgtgttcagcttccccttccaaacctactacctgctggaccagtgggtgttcggcaccgtgatgtgcaaggtggtgtccggcttctactacatcggcttttacagcagcatgttcttcatcaccctgatgagcgtggacagatacctggccgtggtgcatgccgtgtacgctctgaaagtgcggaccatcagaatgggcaccacactgtgtctggctgtgtggctgacagccatcatggccaccattcctctgctggtgttctaccaggtggccagcgaagatggcgtgctgcagtgctacagcttctacaaccagcagaccctgaagtggaagatctttaccaacttcaagatgaatatcctgggcctgctgatccccttcaccatcttcatgttctgctacatcaagatcctgcaccagctgaagcggtgccagaaccacaacaagaccaaggccatccggctggtgctgatcgtcgtgattgcctctctgctgttctgggtgcccttcaacgtggtgctgttcctgacaagcctgcacagcatgcacatcctggacggctgtagcatcagccagcagctgacatacgccacacacgtgaccgagatcatcagcttcacccactgctgcgtgaaccccgtgatctacgcctttgtgggcgagaagttcaagaagcacctgagcgagatctttcagaagtcctgcagccagatcttcaactacctgggcagacagatgcccagagagagctgcgagaagtccagctcctgtcagcagcacagcagcagatctagcagcgtggactatatcctg

SEQ ID NO: 51 - protein, 355 aa, synthetic construct
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 52 - protein, 452 aa, synthetic construct
EVQLLESGGGLVQPGGSLRLSCAASGFTFSSYGMHWVRQAPGKGLEWVSAINWNGGSTGYADSVKGRFTISRDNSKNTLYLQMNSLRAEDTAVYYCARGHHSGYDGRFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 218 aa, synthetic construct
QSVLTQPPSVSGAPGQRVTISCTGSSSNIGAGYNVHWYQQLPGTAPKLLIYTNNRRPSGVPDRFSGSKSGTSASLAISGLRSEDEADYYCAAWDASLSGWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 446 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 219 aa, synthetic construct
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 5 aa, synthetic construct
FYGMA

SEQ ID NO: 57 - protein, 5 aa, synthetic construct
NYGMH

SEQ ID NO: 58 - protein, 5 aa, synthetic construct
DYYMA

SEQ ID NO: 59 - protein, 5 aa, synthetic construct
NYDMA

SEQ ID NO: 60 - protein, 5 aa, synthetic construct
NAAMY

SEQ ID NO: 61 - skipped

SEQ ID NO: 62 - protein, 5 aa, synthetic construct
YYWMT

SEQ ID NO: 63 - protein, 17 aa, synthetic construct
SISYDGSDTYYRDSVKG

SEQ ID NO: 64 - protein, 17 aa, synthetic construct
MIYSDSSKMFYTDTVKG

SEQ ID NO: 65 - protein, 17 aa, synthetic construct
SISYEGRSTYYGDSVKG

SEQ ID NO: 66 - protein, 17 aa, synthetic construct
SVSPSAGTTYYRDSVKG

SEQ ID NO: 67 - protein, 19 aa, synthetic construct
RIRTKPNNYATYYADSVKG

SEQ ID NO: 68 - skipped

SEQ ID NO: 69 - protein, 17 aa, synthetic construct
SITYAGSKTYYGDSVKG

SEQ ID NO: 70 - protein, 17 aa, synthetic construct
SITYAGTKTYYGDSVKG

SEQ ID NO: 71 - protein, 17 aa, synthetic construct
SITNTGDTTYYPDSVKG

SEQ ID NO: 72 - protein, 17 aa, synthetic construct
SISNAGTKTYYGDSVKG

SEQ ID NO: 73 - protein, 17 aa, synthetic construct
SIGYEGSGVYYGDSVKG

SEQ ID NO: 74 - protein, 9 aa, synthetic construct
TSGYGSFDY

SEQ ID NO: 75 - protein, 15 aa, synthetic construct
RDYGYKGAHYYVMDA

SEQ ID NO: 76 - protein, 15 aa, synthetic construct
QDLYYGHKSYWYLDF

SEQ ID NO: 77 - protein, 18 aa, synthetic construct
HMIYRGYNYAVSYYVMDV

SEQ ID NO: 78 - protein, 11 aa, synthetic construct
AVYSSYGYFDY

SEQ ID NO: 79 - skipped

SEQ ID NO: 80 - protein, 15 aa, synthetic construct
QDLYYGHNPYWYFDF

SEQ ID NO: 81 - protein, 15 aa, synthetic construct
QDLYYGRNQYWYLDF

SEQ ID NO: 82 - protein, 15 aa, synthetic construct
QDLYYGRNPSWFLDF

SEQ ID NO: 83 - protein, 11 aa, synthetic construct
GDGGSSFWIAY

SEQ ID NO: 84 - protein, 15 aa, synthetic construct
QDLYYGRNQYWYFDF

SEQ ID NO: 85 - protein, 15 aa, synthetic construct
QELYYGHNPYWYFDF

SEQ ID NO: 86 - protein, 16 aa, synthetic construct
RSSRSLVHRNGDTYLH

SEQ ID NO: 87 - protein, 11 aa, synthetic construct
KSSQNINRYLN

SEQ ID NO: 88 - protein, 11 aa, synthetic construct
KASQNVGSNVD

SEQ ID NO: 89 - protein, 11 aa, synthetic construct
KGSQNINSYLA

SEQ ID NO: 90 - protein, 11 aa, synthetic construct
QASQNNGNWLA

SEQ ID NO: 91 - skipped

SEQ ID NO: 92 - protein, 16 aa, synthetic construct
RSSQSLVHRSGNTYLH

SEQ ID NO: 93 - protein, 11 aa, synthetic construct
KASQSVGSNVD

SEQ ID NO: 94 - protein, 7 aa, synthetic construct
RVSNRFA

SEQ ID NO: 95 - protein, 7 aa, synthetic construct
NANNLQT

SEQ ID NO: 96 - protein, 7 aa, synthetic construct
KTSSRST

SEQ ID NO: 97 - protein, 7 aa, synthetic construct
NTNSLQT

SEQ ID NO: 98 - protein, 7 aa, synthetic construct
GTTNLAD

SEQ ID NO: 99 - skipped

SEQ ID NO: 100 - protein, 7 aa, synthetic construct
KTSSRFT

SEQ ID NO: 101 - protein, 7 aa, synthetic construct
KTTSRYT

SEQ ID NO: 102 - protein, 7 aa, synthetic construct
RVSNRFS

SEQ ID NO: 103 - protein, 9 aa, synthetic construct
LQGTHLPPT

SEQ ID NO: 104 - protein, 9 aa, synthetic construct
LQHKSWPYT

SEQ ID NO: 105 - protein, 9 aa, synthetic construct
MQSNSYPFT

SEQ ID NO: 106 - protein, 10 aa, synthetic construct
YQYNNGYTWT

SEQ ID NO: 107 - protein, 9 aa, synthetic construct
QQASSTPWT

SEQ ID NO: 108 - skipped

SEQ ID NO: 109 - protein, 9 aa, synthetic construct
VQSNSYPYT

SEQ ID NO: 110 - protein, 9 aa, synthetic construct
LQSNSFPYT

SEQ ID NO: 111 - protein, 9 aa, synthetic construct
LQTTHFPPT

SEQ ID NO: 112 - protein, 9 aa, synthetic construct
VQSNSYPYT

SEQ ID NO: 113 - protein, 9 aa, synthetic construct
MQSNSYPYT

SEQ ID NO: 114 - protein, 118 aa, synthetic construct
EVHLVESGGGFVRPGRSMKLSCAASGFPFSFYGMAWVLQTPTKGLEWVASISYDGSDTYYRDSVKGRFAISRDNAKNTLYLQMESLRSEDTATFYCAKTSGYGSFDYWGQGTLVTVSS

SEQ ID NO: 115 - protein, 124 aa, synthetic construct
EVKLVESGGGLVQPGNSLTLSCVASGFTFSNYGMHWIRQAPKKGLEWIAMIYSDSSKMFYTDTVKGRFTISRDNSKNTLYLEMNSLRSEDTAMYYCAARDYGYKGAHYYVMDAWGQGASVTVSS

SEQ ID NO: 116 - protein, 118 aa, synthetic construct
EVQLVESGGGVVQPGRSLRLSCAASGFPFSFYGMAWVRQAPGKGLEWVASISYDGSDTYYRDSVKGRFTISRDNSKNTLYLQMNSLRAEDTAVYYCAKTSGYGSFDYWGQGTLVTVSS

SEQ ID NO: 117 - protein, 124 aa, synthetic construct
EVQLVESGGGVVQPGGSLRLSCAASGFTFSNYGMHWIRQAPGKGLEWIAMIYSDSSKMFYTDTVKGRFTISRDNSKNTLYLQMNSLRAEDTAVYYCAARDYGYKGAHYYVMDAWGQGTTVTVSS

SEQ ID NO: 118 - protein, 123 aa, synthetic construct
EVQLVESGGGLVQPGRSLKLSCVASGFTFSDYYMAWVRQAPKKGLEWVASISYEGRSTYYGDSVKGRFTISRDDAKSTLYLQMNSLRSEDTATYYCARQDLYYGHSYWYLDFWGPGTMVTVSS

SEQ ID NO: 119 - protein, 126 aa, synthetic construct
VQLVESGGGLVQPGRSLKLSCAASGFTFSNYDMAWVRQVPTKGLEWVASVSPSAGTTYYRDSVKGRFTVSRDIAKSSLYLQMDSLRSEDTATYYCARHMIYRGYNYAVSYYVMDVWGQGASVTVSS

SEQ ID NO: 120 - protein, 122 aa, synthetic construct
AVQLVESGGGLVQPKESLKISCAASGFTFSNAAMYWVRQAPGKGLEWVARIRTKPNNYATYYADSVKGRFTISRDDSKSMVYLQMDNLKTEDTAMYYCTAAVYSSYGYFDYWGQGVMVTVSS

SEQ ID NO: 121 - skipped

SEQ ID NO: 122 - protein, 124 aa, synthetic construct
EVQLVESGGGLVQPGRSLKLSCAASGFTFSDYYMAWVRQAPKKGLEWVASITYAGSKTYYGDSVKGRFTISRDDAKSTLYLQMNSLRSEDTATYYCARQDLYYGHNPYWYFDFWGPGTMVTVSS

SEQ ID NO: 123 - protein, 124 aa, synthetic construct
EVQLVESGGGLMQPRRSLKLSCAASGFPFSDYNMAWVRQAPKKGLEWVASITYAGTKTYYGDSVKGRFTISRDDAKSTLYLQMNSLRSEDTATYYCARQDLYYGRNQYWYLDFWGPGTMVTVSS

SEQ ID NO: 124 - protein, 124 aa, synthetic construct
EVQLVESGGGLVQPGRSLKLSCAASGFTFSDYYMAWVRQAPKKGLEWVASISYEGRSTYYGDSVKGRFTISRDDEKSILYLQMSGPRSEDTATYYCARQDLYYGRNPSWFLDFWGPGTMVTVSS

SEQ ID NO: 125 - protein, 120 aa, synthetic construct
EVQLVESGGGLVQPGRSLKLSCVASGFTFNYYWMTWIRQAPGKGLEWVASITNTGDTTYYPDSVKGRFTISRHNAKSTLYLQMNSLRSEDTATYYCSRGDGGSSFWIAYWGQGTLVTVSS

SEQ ID NO: 126 - protein, 124 aa, synthetic construct
EVQLVESGGGLMQPGRSLKLSCAASGFSFSDYYMAWVRQAPKKGLEWVASISNAGTKTYYGDSVKGRFTISRDDAKSTLYLQMNSLRSEDTATYYCVRQDLYYGRNQYWYFDFWGPGTMVTVSS

SEQ ID NO: 127 - protein, 124 aa, synthetic construct
EVQLVESGGGLVQPRRSLKLSCAASGFTFSDYYMAWVRQAPKKGLEWVASIGYEGSGVYYGDSVKGRFTISRDDAKSSLYLQMNSLRSEDTATYYCARQELYYGHNPYWYFDFWGPGTMVTVSS

SEQ ID NO: 128 - protein, 124 aa, synthetic construct
EVQLLESGGGLVQPGGSLRLSCAASGFSFSDYYMAWVRQAPGKGLEWVASISNAGTKTYYGDSVKGRFTISRDDSKNTLYLQMNSLRAEDTAVYYCVRQDLYYGRNQYWYFDFWGRGTLVTVSS

SEQ ID NO: 129 - protein, 112 aa, synthetic construct
DIVMTQSPVSLPVSFGGQASISCRSSRSLVHRNGDTYLHWYLQKPGQSPQLLIYRVSNRFAGVPDRFSGSGSGTDFTLKISRVESEDLGLYYCLQGTHLPPTFGSGTKLEIK

SEQ ID NO: 130 - protein, 107 aa, synthetic construct
DIQMTQSPSFLSASVGDRVTIICKSSQNINRYLNWYQQKLGEAPKLLIYNANNLQTGIPSRFSGGGSGTDFTFTIGSLRPEDVATYFCLQHKSWPYTFGPGTKLELK

SEQ ID NO: 131 - protein, 112 aa, synthetic construct
DIVMTQTPLSLSVTPGQPASISCRSSRSLVHRNGDTYLHWYLQKPGQSPQLLIYRVSNRFAGVPDRFSGSGSGTDFTLKISRVEAEDVGVYYCLQGTHLPPTFGQGTKLEIK

SEQ ID NO: 132 - protein, 107 aa, synthetic construct
DIQMTQSPSSLSASVGDRVTITCKSSQNINRYLNWYQQKPGKAPKLLIYNANNLQTGIPSRFSGSGSGTDFTFTISSLQPEDIATYYCLQHKSWPYTFGQGTKLEIK

SEQ ID NO: 133 - protein, 107 aa, synthetic construct
DIVMTQSPTSMSISVGDRVTMNCKASQNVGSNVDWYQQKPGQSPKLLIYKTSSRSTGVPDRFTGSGSRTDFTFTITNMQAEDLAVYYCMQSNSYPFTFGAGTKLELK

SEQ ID NO: 134 - protein, 107 aa, synthetic construct
IQLTQSPSLLSASVGDRVTLTCKGSQNINSYLAWYQQKLGEAPKLLIYNTNSLQTGIPSRFSGSGSGTDYTLTISSLQPEDVATYFCYQYNNGYTWTFGGGTKLELK

SEQ ID NO: 135 - protein, 107 aa, synthetic construct
DIQMTQSPASLSASLEEIVTITCQASQNNGNWLAWYQQKPGKSPQLLIYGTTNLADGVPSRFSGSRSGTQYSLKISRLQVEDIGIYYCQQASSTPWTFGGGTKLELK

SEQ ID NO: 136 - skipped

SEQ ID NO: 137 - protein, 107 aa, synthetic construct
DIVMTQSPTSMSISVGDRVTMNCKASQNVGSNVDWYQQKTGQSPKLLIYKTSSRFTGVPDRFTGSGSRTDFTFTISNMQAEDLALYYCVQSNSYPYTFGAGTKLELK

SEQ ID NO: 138 - protein, 106 aa, synthetic construct
DIVMTQSPTSMSKSVGDRVTMSCKASQNVGSNVDWYQQKTGQSPKLLIYKTSSRFTGVPDRFTGSGSRTDFTFTISSMQAEDLALYYCVQSNSYPYTFGAGTKLEL

SEQ ID NO: 139 - protein, 106 aa, synthetic construct
IVMTQSPTSMSISVGGRVTMNCKASQNVGSNVDWFQQKTGQSPKLLIYKTTSRYTGVPDRFTGSGSKTDFTFTISNMQAEDLAVYYCLQSNSFPYTFGAGTKLELK

SEQ ID NO: 140 - protein, 112 aa, synthetic construct
DVVMTQTPVSLPVSLGGQASISCRSSQSLVHRSGNTYLHWYLQKPGQSPQLLIYRVSNRFSGGPDRFSGSGSGTTFTLKISRVEPEDLGDYYCLQTTHFPPTFGSGTKLEIK

SEQ ID NO: 141 - protein, 106 aa, synthetic construct
IVMTQSPTSMSKSVGDRVTMNCKASQSVGSNVDWYQQKTGQSPKLLIYKTSSRFTGVPDRFTGSGSRTDFTFTISSMQAEDLALYYCVQSNSYPYTFGAGTKLELK

SEQ ID NO: 142 - protein, 107 aa, synthetic construct
DIVMTQSPTSMSISVGDRVTMNCKASQNVGSNVDWYQQKTGQSPKLLIYKTSSRFTGVPDRFTGSGSKTAFSFTISNMQAEDLAVYYCMQSNSYPYTFGAGTKLELK

SEQ ID NO: 143 - protein, 107 aa, synthetic construct
DIQMTQSPSSLSASVGDRVTITCKASQSVGSNVDWYQQKPGKSPKLLIYKTSSRFTGVPSRFSGSGSRTDFTLTISSLQPEDFATYYCVQSNSYPYTFGAGTKLELK

SEQ ID NO: 144 - protein, 448 aa, synthetic construct
EVHLVESGGGFVRPGRSMKLSCAASGFPFSFYGMAWVLQTPTKGLEWVASISYDGSDTYYRDSVKGRFAISRDNAKNTLYLQMESLRSEDTATFYCAKTSGYGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 454 aa, synthetic construct
EVKLVESGGGLVQPGNSLTLSCVASGFTFSNYGMHWIRQAPKKGLEWIAMIYSDSSKMFYTDTVKGRFTISRDNSKNTLYLEMNSLRSEDTAMYYCAARDYGYKGAHYYVMDAWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 448 aa, synthetic construct
EVQLVESGGGVVQPGRSLRLSCAASGFPFSFYGMAWVRQAPGKGLEWVASISYDGSDTYYRDSVKGRFTISRDNSKNTLYLQMNSLRAEDTAVYYCAKTSGYGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 454 aa, synthetic construct
EVQLVESGGGVVQPGGSLRLSCAASGFTFSNYGMHWIRQAPGKGLEWIAMIYSDSSKMFYTDTVKGRFTISRDNSKNTLYLQMNSLRAEDTAVYYCAARDYGYKGAHYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 454 aa, synthetic construct
EVQLVESGGGLVQPGRSLKLSCVASGFTFSDYYMAWVRQAPKKGLEWVASISYEGRSTYYGDSVKGRFTISRDDAKSTLYLQMNSLRSEDTATYYCARQDLYYGHKSYWYLDFWGPGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 456 aa, synthetic construct
VQLVESGGGLVQPGRSLKLSCAASGFTFSNYDMAWVRQVPTKGLEWVASVSPSAGTTYYRDSVKGRFTVSRDIAKSSLYLQMDSLRSEDTATYYCARHMIYRGYNYAVSYYVMDVWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 452 aa, synthetic construct
AVQLVESGGGLVQPKESLKISCAASGFTFSNAAMYWVRQAPGKGLEWVARIRTKPNNYATYYADSVKGRFTISRDDSKSMVYLQMDNLKTEDTAMYYCTAAVYSSYGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - skipped

SEQ ID NO: 152 - protein, 454 aa, synthetic construct
EVQLVESGGGLVQPGRSLKLSCAASGFTFSDYYMAWVRQAPKKGLEWVASITYAGSKTYYGDSVKGRFTISRDDAKSTLYLQMNSLRSEDTATYYCARQDLYYGHNPYWYFDFWGPGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 454 aa, synthetic construct
EVQLVESGGGLMQPRRSLKLSCAASGFPFSDYNMAWVRQAPKKGLEWVASITYAGTKTYYGDSVKGRFTISRDDAKSTLYLQMNSLRSEDTATYYCARQDLYYGRNQYWYLDFWGPGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 454 aa, synthetic construct
EVQLVESGGGLVQPGRSLKLSCAASGFTFSDYYMAWVRQAPKKGLEWVASISYEGRSTYYGDSVKGRFTISRDDEKSILYLQMSGPRSEDTATYYCARQDLYYGRNPSWFLDFWGPGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 450 aa, synthetic construct
EVQLVESGGGLVQPGRSLKLSCVASGFTFNYYWMTWIRQAPGKGLEWVASITNTGDTTYYPDSVKGRFTISRHNAKSTLYLQMNSLRSEDTATYYCSRGDGGSSFWIAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 454 aa, synthetic construct
EVQLVESGGGLMQPGRSLKLSCAASGFSFSDYYMAWVRQAPKKGLEWVASISNAGTKTYYGDSVKGRFTISRDDAKSTLYLQMNSLRSEDTATYYCVRQDLYYGRNQYWYFDFWGPGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 454 aa, synthetic construct
EVQLVESGGGLVQPRRSLKLSCAASGFTFSDYYMAWVRQAPKKGLEWVASIGYEGSGVYYGDSVKGRFTISRDDAKSSLYLQMNSLRSEDTATYYCARQELYYGHNPYWYFDFWGPGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 454 aa, synthetic construct
EVQLLESGGGLVQPGGSLRLSCAASGFSFSDYYMAWVRQAPGKGLEWVASISNAGTKTYYGDSVKGRFTISRDDSKNTLYLQMNSLRAEDTAVYYCVRQDLYYGRNQYWYFDFWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 219 aa, synthetic construct
DIVMTQSPVSLPVSFGGQASISCRSSRSLVHRNGDTYLHWYLQKPGQSPQLLIYRVSNRFAGVPDRFSGSGSGTDFTLKISRVESEDLGLYYCLQGTHLPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 214 aa, synthetic construct
DIQMTQSPSFLSASVGDRVTIICKSSQNINRYLNWYQQKLGEAPKLLIYNANNLQTGIPSRFSGGGSGTDFTFTIGSLRPEDVATYFCLQHKSWPYTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 219 aa, synthetic construct
DIVMTQTPLSLSVTPGQPASISCRSSRSLVHRNGDTYLHWYLQKPGQSPQLLIYRVSNRFAGVPDRFSGSGSGTDFTLKISRVEAEDVGVYYCLQGTHLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 214 aa, synthetic construct
DIQMTQSPSSLSASVGDRVTITCKSSQNINRYLNWYQQKPGKAPKLLIYNANNLQTGIPSRFSGSGSGTDFTFTISSLQPEDIATYYCLQHKSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 214 aa, synthetic construct
DIVMTQSPTSMSISVGDRVTMNCKASQNVGSNVDWYQQKPGQSPKLLIYKTSSRSTGVPDRFTGSGSRTDFTFTITNMQAEDLAVYYCMQSNSYPFTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 214 aa, synthetic construct
IQLTQSPSLLSASVGDRVTLTCKGSQNINSYLAWYQQKLGEAPKLLIYNTNSLQTGIPSRFSGSGSGTDYTLTISSLQPEDVATYFCYQYNNGYTWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 214 aa, synthetic construct
DIQMTQSPASLSASLEEIVTITCQASQNNGNWLAWYQQKPGKSPQLLIYGTTNLADGVPSRFSGSRSGTQYSLKISRLQVEDIGIYYCQQASSTPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - skipped

SEQ ID NO: 167 - protein, 214 aa, synthetic construct
DIVMTQSPTSMSISVGDRVTMNCKASQNVGSNVDWYQQKTGQSPKLLIYKTSSRFTGVPDRFTGSGSRTDFTFTISNMQAEDLALYYCVQSNSYPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 213 aa, synthetic construct
DIVMTQSPTSMSKSVGDRVTMSCKASQNVGSNVDWYQQKTGQSPKLLIYKTSSRFTGVPDRFTGSGSRTDFTFTISSMQAEDLALYYCVQSNSYPYTFGAGTKLELRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 213 aa, synthetic construct
IVMTQSPTSMSISVGGRVTMNCKASQNVGSNVDWFQQKTGQSPKLLIYKTTSRYTGVPDRFTGSGSKTDFTFTISNMQAEDLAVYYCLQSNSFPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 219 aa, synthetic construct
DVVMTQTPVSLPVSLGGQASISCRSSQSLVHRSGNTYLHWYLQKPGQSPQLLIYRVSNRFSGGPDRFSGSGSGTTFTLKISRVEPEDLGDYYCLQTTHFPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 213 aa, synthetic construct
IVMTQSPTSMSKSVGDRVTMNCKASQSVGSNVDWYQQKTGQSPKLLIYKTSSRFTGVPDRFTGSGSRTDFTFTISSMQAEDLALYYCVQSNSYPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 214 aa, synthetic construct
DIVMTQSPTSMSISVGDRVTMNCKASQNVGSNVDWYQQKTGQSPKLLIYKTSSRFTGVPDRFTGSGSKTAFSFTISNMQAEDLAVYYCMQSNSYPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 214 aa, synthetic construct
DIQMTQSPSSLSASVGDRVTITCKASQSVGSNVDWYQQKPGKSPKLLIYKTSSRFTGVPSRFSGSGSRTDFTLTISSLQPEDFATYYCVQSNSYPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 443 aa, synthetic construct
QVQLQESGPGLMKPSETLSLTCSVSGDSIRSDYWSWIRQPPGKGLEYIGYVSYSGSTYYNPSLKSRVTISVDTSKNRFSLKLNSVTAADTAVYYCARWDGDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 214 aa, synthetic construct
EIVMTQSPATLSVSPGERATLSCRASQSIGNNLHWYQQKPGQAPRLLIYYASQSISGIPARFSGSGSGTEFTLTISSLQSEDFAVYYCQQSNSWPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 451 aa, synthetic construct
EVQLVESGGGLVQPGGSLKLSCAASGFTFNTYAMNWVRQASGKGLEWVGRIRSKSNNYATYYADSVKDRFTISRDDSKNTAYLQMNSLKTEDTAVYYCVRGLLRYRFFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 219 aa, synthetic construct
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 453 aa, synthetic construct
EVQLVETGGGLVQPKGSLRLSCAASGFPFNTFAMNWVRQAPGKGLEWLARIRSKSNNYATYYADSVKDRFTISRDDSRRMLYLLMNNVKFEDTAIYYCVRGKENNYGYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 219 aa, synthetic construct; identical to SEQ ID NO: 40